(19) United States
(12) Patent Application Publication
(10) Pub. No.: US 2019/0078133 A1
(43) Pub. Date: Mar. 14, 2019

(54) DETECTION AND IDENTIFICATION OF BACTERIA AND DETERMINATION OF ANTIBIOTIC SUSCEPTIBILITY USING BACTERIOPHAGE AND REPORTER MOLECULES

(71) Applicant: The Charles Stark Draper Laboratory, Inc., Cambridge, MA (US)

(72) Inventors: Peter Cavanagh, Concord, MA (US); Quin Christensen, Cambridge, MA (US); Kenneth T. Kotz, Cambridge, MA (US); Jason Holder, Swampscott, MA (US)

(21) Appl. No.: 16/123,875

(22) Filed: Sep. 6, 2018

Related U.S. Application Data
(60) Provisional application No. 62/556,108, filed on Sep. 8, 2017.

(51) Int. Cl. C12Q 1/10; C12Q 1/18; C12N 7/00

(52) U.S. Cl. CPC C12Q 1/10; C12N 2795/10231; C12N 7/00; C12Q 1/18

(57) ABSTRACT

The present disclosure provides compositions and methods for identifying bacteria and profiling their antibiotic susceptibility. In particular, the methods and compositions of the present technology permit the detection of low concentrations of bacterial cells (e.g., <10 cells/ml) that are present within a complex biological sample.

### CROSS-REFERENCES TO RELATED APPLICATIONS

This application claims priority under 35 U.S.C. § 119(e) to U.S. Provisional Application No. 62/556,108, filed Sep. 8, 2017, which is incorporated by reference in its entirety.

### TECHNICAL FIELD

The present technology relates generally to compositions and methods for identifying bacteria and profiling their antibiotic susceptibility

### BACKGROUND

The following description of the background of the present technology is provided simply as an aid in understanding the present technology and is not admitted to describe or constitute prior art to the present technology.

Bacterial infections may complicate a patient's existing medical condition, and in some cases, may lead to death. Determining the presence of a bacterial infection as well as the identity of the infection within an hour of isolating a biological sample is critical for improved patient outcomes. Patients suffering from various bacterial infections often present with similar symptoms, thus making it difficult to accurately identify and characterize the bacterial species or strain responsible for the infection. Accurate identification of the bacteria through conventional lab tests can be challenging and may require incubation periods of up to several days. Additionally, some bacterial strains are not amenable to culturing and in vitro analysis in light of their fastidious nature. In other situations, the observable behavior of some bacterial strains is not readily distinguishable from others.

Moreover, components of whole blood can also interfere with the detection of bacterial cells. For example, white blood cells are capable of immobilizing, inactivating, or killing microbes, thus interfering with the detection of pathogenic bacteria in blood. Other blood components may outnumber bacteria within a sample, making enumeration of bacteria within the blood sample difficult. For example, there are approximately 50-400×10⁶ platelets in one ml of whole blood. Platelets are similar in size to bacteria thus rendering sized-based counting almost impossible. Red blood cells, which have a strong optical absorption in the visible spectrum due to hemoglobin, can confound or attenuate many of the standard absorption, luminescent, or fluorescence based assays. Sample processing methods are generally required to accurately measure clinically relevant concentrations (e.g., 10 cells/ml) of bacteria within whole blood.

Early and accurate identification of the bacterial strain(s) responsible for a patient's illness and determining its susceptibility to various antibiotics is an important aspect of the treatment selection decision process. Effective treatment requires knowledge of the antibiotic susceptibility of a particular bacterial species. Conventional antibiotic susceptibility testing requires additional sample processing steps, increasing testing costs and culture time periods upwards of 24-48 additional hours. To date none of the existing methods used in clinical practice are capable of detecting rare bacterial cells in whole blood within a short time frame. The combination of sensitivity, specificity, sample processing, and time requirements pose an enormous challenge to developing effective diagnostic methods for detecting bacterial infections.

### SUMMARY OF THE PRESENT TECHNOLOGY

In one aspect, the present disclosure provides a labelled detector bacteriophage (LDB) that includes a capsid and nucleic acids wherein the capsid comprises an exterior surface relative to the nucleic acids of the phage; and a labelling moiety, wherein the labelling moiety is covalently linked to the exterior surface of the phage via an amide group or groups. The labelling moiety may be a bioluminescent moiety, a fluorescent moiety, or a chromogenic moiety.

The amide group or groups of the LDB may include

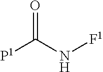

wherein P¹ represents the phage, the C═O is a carbonyl group of the phage, F¹ represents the labelling moiety, and N is a nitrogen of an amino group of the labelling moiety. In any embodiment herein, the amide group or groups may include an aspartic acid and/or a glutamic acid residue on the exterior surface of the capsid. The amide group or groups may include

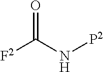

wherein P² represents the phage, N is a nitrogen of an amino group of the phage, F² represents the labelling moiety, and C═O is a carbonyl group of the labelling moiety. In any embodiment herein, the amide group or groups may include a lysine residue of the bacteriophage, such as a lysine residue on the exterior surface of the capsid. In any embodiment herein, the amide group or groups may include a linker of formula

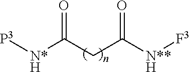

where P³ represents the phage, N* is a nitrogen of an amino group of the phage (e.g., a lysine residue on the exterior surface of the capsid), F³ represents the labelling moiety, N** is a nitrogen of an amino group of the labelling moiety, and n is 1, 2, 3, 4, 5, 6, 7, 8, 9, or 10.

The LDB may include two or more labelling moieties, wherein each labelling moiety is independently covalently linked to the exterior surface of the phage via an amide group or groups. The labelling moieties may include a bioluminescent moiety, a fluorescent moiety, a chromogenic moiety, or a combination of any two or more thereof.

In related aspects, methods are provided for generating a LDB.

In one aspect, the present disclosure provides a method for identifying at least one bacterial strain or species in a biological sample obtained from a subject comprising (a) contacting the biological sample with an effective amount of any of the above described LDB; and (b) detecting the presence of LDB-infected bacterial cells, thereby leading to the identification of at least one bacterial strain or species in the biological sample.

In one aspect, the present disclosure provides a method for determining the antibiotic susceptibility of a bacterial strain or species in a biological sample obtained from a subject comprising (a) contacting a plurality of test samples comprising bacterial cells with an effective amount of any of the above described LDB and at least one antibiotic, wherein the plurality of test samples is derived from the subject; (b) detecting the presence of LDB-infected bacterial cells in the plurality of test samples; and (c) determining that an antibiotic is effective in inhibiting the bacterial strain or species when the number of LDB-infected bacterial cells in the test sample is reduced relative to that observed in an untreated control sample comprising bacterial cells, wherein the untreated control sample is derived from the subject. In some embodiments, the antibiotic susceptibility of the bacterial strain or species is determined within 15 minutes after contacting the biological sample with the LDB and the at least one antibiotic.

Additionally or alternatively, in some embodiments of the methods disclosed herein, the LDB further comprises at least one reporter molecule that is intercalated within the nucleic acids of the LDB. Examples of nucleic acid intercalating reporter molecules include, but are not limited to SYBR Gold, SYBR Green I, SYBR Safe, Quant-iT PicoGreen, Blue-Fluorescent SYTO dyes (e.g., SYTO 40, SYTO 41, SYTO 42, SYTO 45), Green-Fluorescent SYTO dyes (e.g., SYTO 9, SYTO 10, SYTO BC, SYTO 13, SYTO 16, SYTO 24, SYTO 21, SYTO 12, SYTO 11, SYTO 14, SYTO 25), Orange-Fluorescent SYTO dyes (e.g., SYTO 81, SYTO 80, SYTO 82, SYTO 83, SYTO 84, SYTO 85), Red-Fluorescent SYTO dyes (e.g., SYTO 64, SYTO 61, SYTO 17, SYTO 59, SYTO 62, SYTO 60, SYTO 63), cyanine dimer dyes (e.g., TOTO-1, YOYO-1, POPO-1, LOLO-1, BOBO-1, JOJO-1, POPO-3, BOBO-3, YOYO-3, TOTO-3), and other cyanine dyes, Acridine homodimer, Acridine orange, 7-AAD, ACMA, DAPI, Dihydroethidium, ethidium bromide, EthD-1, EthD-2, Hoechst 33258, Hoechst 33342, Hoechst 34580, hydroxystilbamidine, and LDS 751.

Additionally or alternatively, in some embodiments of the methods disclosed herein, the LDB further comprises at least one reporter molecule that is encoded by a heterologous nucleic acid located within the genome of the LDB. The at least one reporter molecule encoded by the heterologous nucleic acid may be a fluorescent label, a luminescent label, a colorimetric label, an electrochemical label, or a mechanical label. In some embodiments, the fluorescent label is a fluorescent protein selected from the group consisting of TagBFP, Azurite, EBFP2, mKalamal, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, and Dronpa. In certain embodiments, the luminescent label is a bioluminescent protein selected from the group consisting of Aequorin, firefly luciferase, Renilla luciferase, red luciferase, luxAB, and nanoluciferase. In some embodiments, the colorimetric label is a chemiluminescent protein selected from the group consisting of β-galactosidase, horseradish peroxidase (HRP), and alkaline phosphatase.

Additionally or alternatively, in some embodiments, the at least one reporter molecule encoded by the heterologous nucleic acid may be captured on a microbead or a solid support microstructure. In certain embodiments, the at least one reporter molecule encoded by the heterologous nucleic acid comprises an affinity domain that specifically binds to the microbead or the solid support microstructure. Additionally or alternatively, in some embodiments of the methods disclosed herein, the microbead or the solid support microstructure is coated with a reagent that allows capture of the at least one reporter molecule produced by LDB-infected bacterial cells.

In certain embodiments, the methods of the present technology further comprise contacting the LDB-infected bacterial cells with a microbead or a solid support microstructure that is optionally coated with a reagent that allows capture of the LDB-infected bacterial cells. The microbead or solid support microstructure may be coded to facilitate the identification of a specific bacteria strain or species in the biological sample.

Additionally or alternatively, in some embodiments of the methods disclosed herein, the microbead or the solid support microstructure is captured in a microwell array that is optionally sealed by mechanical sealing or oil sealing. The microwell array may contain 1 bead per well or more than 1 bead/well.

In certain embodiments of the methods disclosed herein, the microbead or the solid support microstructure is spread on a surface that does not contain microwells.

In any of the above embodiments of the methods disclosed herein, the presence of LDB-infected bacterial cells is detected within about 5 to 90 minutes after contacting the biological sample with the LDB.

Additionally or alternatively, in some embodiments of the methods disclosed herein, the biological sample comprises no more than 10 bacterial cells/ml, about 10 to 20 bacterial cells/ml, about 5 to 50 bacterial cells/ml, about 50 to 400 bacterial cells/ml, about 20 to 300 bacterial cells/ml, about 30 to 500 bacterial cells/ml, about 40 to 200 bacterial cells/ml, or about 50 to 450 bacterial cells/ml.

Also disclosed herein are kits comprising one or more coded/labeled vials that contain a plurality of the labelled detector bacteriophages disclosed herein, and instructions for use.

### BRIEF DESCRIPTION OF THE DRAWINGS

FIG. 1 is a schematic of four different embodiments of methods for detecting and antibiotic susceptibility profiling of bacteria using labelled detector bacteriophages of the present technology in microwell assays.

FIG. 2 is a schematic of recombinant bacteriophages that are engineered to produce a recombinant reporter protein that comprises an active domain of an enzyme and an affinity tag domain that is useful for subsequent bead-based capture of the recombinant reporter protein.

FIG. 3 is a schematic of bead-based capture assays in microwells used to capture bacteriophage-produced recombinant reporter proteins or bacteria infected with bacteriophages comprising the recombinant reporter protein.

FIG. 4A shows a method for labelling the surface of the T7 phage with FITC via an alkaline-buffered condensation reaction of NHS-ester-FITC and the primary amines (—NH₂) of lysine residues on the surface of the T7 phage. FIG. 4B shows that fluorescence was detected in the infected TOP10b cells (a strain of Escherichia coli (E. coli) cells), whereas no fluorescence was detected in either K5 cells (another E. coli strain) or the Klebsiella pneumoniae (K. pneumoniae) strain Kp 390 cells. TOP10b cells (New England Biolabs, Ipswich, Mass.) are normally targeted by T7 phage, whereas K5 cells and Kp 390 cells are not host cells for T7 phage. TOP10b cells, K5 cells, and Kp 390 cells were infected with FITC labelled T7 bacteriophages. The FITC labelled T7 phages were incubated with the bacterial cells at a multiplicity of infection (MOI) of 100 phage:1 bacterial cell.

FIG. 5A shows phages that were labelled with different fluorophores using varying coupling chemistries. The coupling chemistries were carried out for 12 hours (until completion) and the resulting phages were purified away from the unincorporated dye by dialysis for the smaller organic dyes, and by DEAE anion exchange chromatography for the Qdot (Thermo Fisher Scientific, Cambridge, Mass.) labelled phages. FIG. 5B shows the results for final titer, final fluorescence, and total activity for each labelled phage described in FIG. 5A. For each labelled phage, the fluorophore concentration goes from the highest value to the lowest value (left to right). Total activity was calculated by multiplying the titer by the total fluorescence.

FIG. 6A shows that fluorescence was detected in E. coli TOP10b cells infected with Hoechst labelled T7 bacteriophages. TOP10b cells were infected with Hoechst labelled T7 bacteriophages at a MOI of 100 phage:1 bacterial cell for 20 minutes. FIG. 6B shows that fluorescence was detected in TOP10b cells infected with SYBR Gold labelled T7 bacteriophages. TOP10b cells were infected with SYBR Gold labelled T7 bacteriophages at a MOI of 100 phage:1 bacterial cell for 20 minutes.

FIG. 7 shows the multispecies detection of E. coli TOP10b cells and Pseudomonas aeruginosa (P. aeruginosa) strain A1 cells in a sample. TOP10b cells infected with Hoechst labelled T7 phages produced a fluorescent signal (marked with gray arrows). A1 cells infected with SYBR Gold labelled PB1 phages produced a fluorescent signal (marked with white arrows).

FIG. 8 shows an exemplary method for detecting bacteria in an unpurified sample using labelled detector bacteriophages of the present technology in microwells.

FIG. 9 shows an exemplary method for detecting bacteria in a purified sample using labelled detector bacteriophages of the present technology in microwells.

FIG. 10 shows an exemplary method of antibiotic susceptibility profiling in an unpurified sample using labelled detector bacteriophages of the present technology in microwells.

FIG. 11 shows an exemplary method of antibiotic susceptibility profiling in a purified sample using labelled detector bacteriophages of the present technology in microwells.

FIG. 12 shows an exemplary method of detecting, enumerating and antibiotic susceptibility profiling in an unpurified sample using labelled detector bacteriophages of the present technology in microwells.

FIG. 13 shows an exemplary method of detecting, enumerating and antibiotic susceptibility profiling in a purified sample using labelled detector bacteriophages of the present technology in microwells.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Effective methods for bacterial identification and antibiotic susceptibility profiling must recognize only living bacterial cells. The specific recognition of living bacterial cells is critical for the determination of antibiotic susceptibility. The present disclosure provides methods and compositions for the identification of bacterial strains present within blood or other clinical samples, such as tissues, swabs, or other biofluids. The present disclosure also provides methods for determining the antibiotic susceptibility profile of bacterial strains. The methods disclosed herein show superior sensitivity and specificity compared to conventional techniques for bacterial identification and antibiotic susceptibility profiling. For example, the methods and compositions of the present technology permit the detection of low concentrations of bacterial cells (e.g., less than 10 cells/ml) that are present within a complex biological sample and shortens the time required to obtain results compared to conventional methods. The present technology also provides methods and compositions for detecting multiple bacterial species/strains from a single sample. The methods disclosed herein can be used in a variety of applications including monitoring bacterial growth in water and food, veterinary diagnostics, or screening for new antibiotics.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “μ” is the signal separation between untreated bacterial host cells infected with a labelled detector phage and antibiotic-treated bacterial host cells infected with a labelled detector phage (μ=signal of untreated labelled detector phage-infected host cells/signal of antibiotic treated labelled detector phage-infected host cells). In some embodiments, a μ greater than or equal to 2 is indicative of antibiotic sensitivity for a given bacterial host.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “amide” (or “amido”) includes C- and N-amide groups, i.e., —C(O)NR⁷¹R⁷², and —NR⁷¹C(O)R⁷² groups, respectively. For example, R⁷¹ and R⁷² may each independently be hydrogen, or a substituted or unsubstituted alkyl, alkenyl, alkynyl, cycloalkyl, aryl, aralkyl, heterocyclylalkyl, or heterocyclyl group.

The term “amine” (or “amino”) as used herein refers to —NR⁷⁵R⁷⁶ groups. For example, R⁷⁵ and R⁷⁶ may independently be hydrogen, or a substituted or unsubstituted alkyl, alkenyl, alkynyl, cycloalkyl, aryl, aralkyl, heterocyclylalkyl, or heterocyclyl group. A “primary amine” refers to an —NH₂ group on a compound.

As used herein, “bacteriophage” or “phage” refers to a virus that infects bacteria. Bacteriophages are obligate intracellular parasites that multiply inside bacteria by co-opting some or all of the host biosynthetic machinery (i.e., viruses that infect bacteria). Though different bacteriophages may contain different materials, they all contain nucleic acid and protein, and can under certain circumstances be encapsulated in a lipid membrane. Depending upon the phage, the nucleic acid can be either DNA or RNA (but not both).

The term “carboxyl group” as used herein refers to a compound with a —C(O)OH or —C(O)O⁻ group.

As used herein, the term “effective amount” refers to a quantity sufficient to achieve a desired effect, e.g., an amount of a labelled detector bacteriophage which results in the identification of bacteria and/or determination of antibiotic susceptibility. The amount of a labelled detector bacteriophage contacted with a sample will depend on the degree, type, and severity of the bacterial infection. The skilled artisan will be able to determine appropriate dosages depending on these and other factors.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, “heterologous nucleic acid sequence” is any sequence placed at a location in the genome where it does not normally occur. A heterologous nucleic acid sequence may comprise a sequence that does not naturally occur in a bacteriophage, or it may comprise only sequences naturally found in the bacteriophage, but placed at a non-normally occurring location in the genome. In some embodiments, the heterologous nucleic acid sequence is not a natural phage sequence. In certain embodiments, the heterologous nucleic acid sequence is a natural phage sequence that is derived from a different phage. In other embodiments, the heterologous nucleic acid sequence is a sequence that occurs naturally in the genome of a wild-type phage but is then relocated to another site where it does not naturally occur, rendering it a heterologous sequence at that new site.

As used herein, a “host cell” is a bacterial cell that can be infected by a phage to yield progeny phage particles. A host cell can form phage particles from a particular type of phage genomic DNA. In some embodiments, the phage genomic DNA is introduced into the host cell by infecting the host cell with a phage. In some embodiments, the phage genomic DNA is introduced into the host cell using transformation, electroporation, or any other suitable technique. In some embodiments, the phage genomic DNA is substantially pure when introduced into the host cell. In some embodiments, the phage genomic DNA is present in a vector when introduced into the host cell. The definition of host cell can vary from one phage to another. For example, E. coli may be the natural host cell for a particular type of phage, but Klebsiella pneumoniae is not.

As used herein, “host range” refers to the ranges of bacteria that are infected by a particular bacteriophage. The bacterial hosts often are related phylogenetically, and share some similar chemical features on the surface of the bacterial cells.

As used herein, the terms “individual”, “patient”, or “subject” are used interchangeably and refer to an individual organism, a vertebrate, a mammal, or a human. In certain embodiments, the individual, patient or subject is a human.

As used herein, the term “isolated” refers to a substance or entity that has been separated from at least some of the components with which it was associated when initially produced (whether in nature or in an experimental setting). Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances and/or entities are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups as well as procedures to add or remove such protecting groups may be found in Greene, T. W.; Wuts, P. G. M. (1999) Protective Groups in Organic Synthesis, 3rd Ed. (John Wiley & Sons, Inc., New York), hereby incorporated by reference in its entirety and for any and all purposes as if fully set forth herein. For example, amino protecting groups include, but are not limited to, mesitylenesulfonyl (Mts), benzyloxycarbonyl (Cbz or Z), 2-chlorobenzyloxycarbonyl, t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α-,α-dimethyldimethoxybenzyloxycarbonyl (DDZ), 5-bromo-7-nitroindolinyl, and the like. As another example, carboxyl groups may be protected as an ester.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the genome of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, a “labelled detector bacteriophage” or “labelled detector phage” or “LDB” means a bacteriophage that comprises a bioluminescent label, a fluorescent label, a chromogenic label, or any combination thereof.

As used herein, a “recombinant bacteriophage genome” is a bacteriophage genome that has been genetically modified by the insertion of a heterologous nucleic acid sequence into the bacteriophage genome. A “recombinant bacteriophage” means a bacteriophage that comprises a recombinant bacteriophage genome. In some embodiments, the bacteriophage genome is modified by recombinant DNA technology to introduce a heterologous nucleic acid sequence into the genome at a defined site. In some embodiments, the heterologous nucleic acid sequence is introduced with no corresponding loss of endogenous phage genomic nucleotides. In other words, if bases N1 and N2 are adjacent in the wild-type bacteriophage genome, the heterologous nucleic acid sequence is inserted between N1 and N2. Thus, in the resulting recombinant bacteriophage genome, the heterologous nucleic acid sequence is flanked by nucleotides N1 and N2. In some embodiments, endogenous phage nucleotides are removed or replaced during the insertion of the heterologous nucleic acid sequence. For example, in some embodiments, the heterologous nucleic acid sequence is inserted in place of some or all of the endogenous phage sequence which is removed. In some embodiments, endogenous phage sequences are removed from a position in the phage genome distant from the site(s) of insertion of the heterologous nucleic acid sequences.

As used herein, the term “sample” refers to clinical samples obtained from a subject or isolated microorganisms. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, a “test sample” refers to a sample taken from a subject that is to be assayed for the presence of bacteria and/or for the antibiotic susceptibility of bacteria present in the sample. In some embodiments, the test sample is blood, sputum, mucus, lavage, or saliva. In some embodiments, the test sample is a swab from a subject.

### Bacteriophage

Bacteriophage are obligate intracellular parasites that multiply inside bacteria by co-opting some or all of the host biosynthetic machinery. Phages contain nucleic acid and protein, and may be enveloped by a lipid membrane. Depending upon the phage, the nucleic acid genome can be either DNA or RNA but not both, and can exist in either circular or linear forms. The size of the phage genome varies depending upon the phage. The simplest phages have genomes that are only a few thousand nucleotides in size, while the more complex phages may contain more than 100,000 nucleotides in their genome, and in rare instances more than 1,000,000. The number and amount of individual types of protein in phage particles will vary depending upon the phage. The proteins function in infection and to protect the nucleic acid genome from environmental nucleases.

Phage genomes come in a variety of sizes and shapes (e.g., linear or circular). Most phages range in size from 24-200 nm in diameter. The capsid is composed of many copies of one or more phage proteins, and acts as a protective envelope around the phage genome. Many phages have tails attached to the phage capsid. The tail is a hollow tube through which the phage nucleic acid passes during infection. The size of the tail can vary and some phages do not even have a tail structure. In the more complex phages, the tail is surrounded by a contractile sheath which contracts during infection of the bacterial host cell. At the end of the tail, phages have a base plate and one or more tail fibers attached to it. The base plate and tail fibers are involved in the binding of the phage to the host cell.

Lytic or virulent phages are phages which can only multiply in bacteria and lyse the bacterial host cell at the end of the life cycle of the phage. The lifecycle of a lytic phage begins with an eclipse period. During the eclipse phase, no infectious phage particles can be found either inside or outside the host cell. The phage nucleic acid takes over the host biosynthetic machinery and phage specific mRNAs and proteins are produced. Early phage mRNAs code for early proteins that are needed for phage DNA synthesis and for shutting off host DNA, RNA and protein biosynthesis. In some cases, the early proteins actually degrade the host chromosome. After phage DNA is made late mRNAs and late proteins are made. The late proteins are the structural proteins that comprise the phage as well as the proteins needed for lysis of the bacterial cell. In the next phase, the phage nucleic acid and structural proteins are assembled and infectious phage particles accumulate within the cell. The bacteria begin to lyse due to the accumulation of the phage lysis protein, leading to the release of intracellular phage particles. The number of particles released per infected cell can be as high as 1000 or more. Lytic phage may be enumerated by a plaque assay. The assay is performed at a low enough concentration of phage such that each plaque arises from a single infectious phage. The infectious particle that gives rise to a plaque is called a PFU (plaque forming unit).

Lysogenic phages are those that can either multiply via the lytic cycle or enter a quiescent state in the host cell. In the quiescent state, the phage genome exists as a prophage (i.e., it has the potential to produce phage). In most cases, the phage DNA actually integrates into the host chromosome and is replicated along with the host chromosome and passed on to the daughter cells. The host cell harboring a prophage is not adversely affected by the presence of the prophage and the lysogenic state may persist indefinitely. The lysogenic state can be terminated upon exposure to adverse conditions. Conditions which favor the termination of the lysogenic state include: desiccation, exposure to UV or ionizing radiation, exposure to mutagenic chemicals, etc. Adverse conditions lead to the production of proteases (rec A protein), the expression of the phage genes, reversal of the integration process, and lytic multiplication.

In some embodiments, a phage genome comprises at least 5 kilobases (kb), at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, at least 80 kb, at least 85 kb, at least 90 kb, at least 95 kb, at least 100 kb, at least 105 kb, at least 110 kb, at least 115 kb, at least 120 kb, at least 125 kb, at least 130 kb, at least 135 kb, at least 140 kb, at least 145 kb, at least 150 kb, at least 175 kb, at least 200 kb, at least 225 kb, at least 250 kb, at least 275 kb, at least 300 kb, at least 325 kb, at least 350 kb, at least 375 kb, at least 400 kb, at least 425 kb, at least 450 kb, at least 475 kb, or at least 500 kb of nucleic acids.

Phage groups include Myoviridae, Siphoviridae, Podoviridae, Lipothrixviridae, Rudiviridae, Ampullaviridae, Bucaudaviridae, Clavaviridae, Corticoviridae, Cystoviridae, Fuselloviridae, Globuloviriade, Guttaviridae, Inoviridae, Leviviridae, Mircoviridae, Plasmaviridae, and Tectiviridae.

### Labelled Phage Compositions of the Present Technology

The present disclosure provides bacteriophage compositions that are useful as a label to detect the presence of host bacterial cells that the phage specifically infect. In certain embodiments, one or more reporter molecules may be attached to the exterior surface of the bacteriophage capsid via various coupling chemistries. Typically hundreds to thousands of phage will attach to a bacteria host cell when the bacterial host cell is infected with phage particles at a high multiplicity of infection (MOI). Over the course of the infection, the reporter molecules are spatially concentrated around the target bacterial cells, which can then be detected using imaging microscopy, biochemical assays, or flow cytometry.

In one aspect, the present disclosure provides a labelled detector bacteriophage (LDB) that includes a capsid and nucleic acids wherein the capsid comprises an exterior surface relative to the nucleic acids of the phage; and a labelling moiety, wherein the labelling moiety is covalently linked to the exterior surface of the phage via an amide group or groups. For example, two or more labelling moieties may each independently be covalently linked to the exterior surface of the phage by independent amide groups. The capsid of the phage may comprise a two or more amines, two or more carboxyl groups, or a combination thereof; the capside of the phage may comprise a plurality of amines, a plurality of carboxyl groups, or a combination thereof. The amide groups are originally from a carboxyl group and/or amine group of the phage, where the amine and/or carboxyl group were disposed on the exterior surface of the capsid prior to forming an amide bond with a labelling moiety. The labelling moiety may be a bioluminescent moiety, a fluorescent moiety, or a chromogenic moiety.

The amide group or groups of the LDB may include

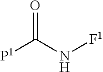

wherein P¹ represents the phage, the C═O is a carbonyl group of the phage, F¹ represents the labelling moiety, and N is a nitrogen of an amino group of the labelling moiety. In any embodiment herein, the amide group or groups may be derived from an aspartic acid and/or a glutamic acid residue on the exterior surface of the capsid. The amide group or groups may include

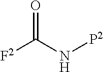

wherein P² represents the phage, N is a nitrogen of an amino group of the phage, F² represents the labelling moiety, and C═O is a carbonyl group of the labelling moiety. In any embodiment herein, the amide group or groups may be derived from a lysine residue of the bacteriophage, such as a lysine residue on the exterior surface of the capsid. In any embodiment herein, the amide group or groups may include a linker of formula

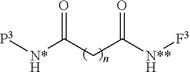

where P³ represents the phage, N* is a nitrogen of an amino group of the phage (e.g., a lysine residue on the exterior surface of the capsid), F³ represents the labelling moiety, N** is a nitrogen of an amino group of the labelling moiety, and n is 1, 2, 3, 4, 5, 6, 7, 8, 9, or 10.

As discussed above, the LDB may include two or more labelling moieties, wherein each labelling moiety is independently covalently linked to the exterior surface of the phage via an amide group or groups. The labelling moieties may include a bioluminescent moiety, a fluorescent moiety, a chromogenic moiety, or a combination of any two or more thereof. In any embodiment herein, the labelling moiety or moieties of the LDB may include one or more fluorescent moieties such as Atto Dye 590, Atto Dye 594, an amine- or carboxylate-functionalized quantum dot (e.g., amine-funcationalized Q-Dot 545, amine-funcationalized Q-Dot 585, amine-funcationalized Q-Dot 605, amine-funcationalized Q-Dot 655, amine-funcationalized Q-Dot 705, amine-funcationalized Q-Dot 800, carboxylate-funcationalized Q-Dot 545, carboxylate-funcationalized Q-Dot 585, carboxylate-funcationalized Q-Dot 605, carboxylate-funcationalized Q-Dot 655, carboxylate-funcationalized Q-Dot 705, carboxylate-funcationalized Q-Dot 800), fluorescein isothiocyanate (FITC), 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); VIC®, or a combination of any two or more thereof.

In a related aspect, the present disclosure provides a methods for generating a LDB where the amide group or groups of the LDB include one or both of

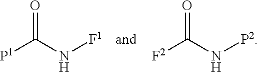

In some embodiments of the method, method includes contacting a phage that includes a carboxyl group disposed on the exterior surface of the capsid, a first activating agent, and a first carbodiimmide in the presence of a first solvent (that includes water) to provide a first activated species; and subsequently contacting a first labelling moiety that includes an amine group with the first activated species to provide a first LDB. The method may further include generating one or more amine groups disposed on the exterior surface of the capsid of the first LDB; contacting a second labelling moiety comprising a carboxyl group, a second activating agent, and a second carbodiimmide in the presence of a second solvent (that includes water) to provide a second activated species; subsequent to the generating, contacting the first LDB with the second activated species to provide a second LDB; where the first labelling moiety and the second labelling moiety are each independently covalently linked to the exterior surface of the second LDB via an amide group or groups. Generating one or more amine groups may include removing a protecting group from one or more protected amino groups of the first LDB. The method may include contacting the first LDB (subsequent to the generating step) or the second LDB with a third labelling moiety comprising an amine group and an activated linker of the formula

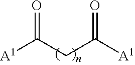

in the presence of a third solvent (that includes water) to provide a third LDB, wherein A¹ is a heteroatom of a third activating agent.

In some embodiments of the method, method includes contacting a first labelling moiety comprising a carboxyl group, a first activating agent, and a first carbodiimmide in the presence of a first solvent (that includes water) to provide a first activated species; and subsequently contacting a phage that includes an amine group disposed on the exterior surface of a capsid of the phage with the first activated species to provide a first LDB. The method may further include generating one or more carboxyl groups disposed on the exterior surface of the capsid of the first LDB; subsequently contacting the first LDB, a second activating agent, and a second carbodiimmide in the presence of a second solvent (that includes water) to provide a second activated species; and contacting a second labelling moiety comprising an amine group with the second activated species to provide a second LDB; where the first labelling moiety and the second labelling moiety are each independently covalently linked to the exterior surface of the second LDB via an amide group or groups. Generating one or more carboxyl groups may include removing a protecting group from one or more protected carboxyl groups of the first LDB. The method may include optionally generating one or more amine groups disposed on the exterior surface of the capsid of the first LDB or the second LDB; contacting the first LDB comprising one or more amine groups disposed on the exterior surface of the capsid or the second LDB (subsequent to the optional generating) with a third labelling moiety comprising an amine group and an activated linker of the formula

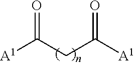

in the presence of a third solvent (that includes water) to provide a third LDB, wherein A¹ is a heteroatom of a third activating agent.

In any embodiment herein, a pH of the contacting step including the first solvent may be about 5.5 to about 6.5 and the contacting step further comprises adjusting the pH of the first solvent to about 7 to about 9 subsequent to providing the first activated species. In any embodiment herein, a pH of the contacting step including the second solvent may be about 5.5 to about 6.5 and the contacting step further comprises adjusting the pH of the first solvent to about 7 to about 9 subsequent to providing the second activated species. A pH of the contacting step including the third solvent may be about 7 to about 9. The pH of the first solvent and the second solvent prior to adjusting the pH may independently be for each solvent about 5.5, about 5.6, about 5.7, about 5.8, about 5.9, about 6.0, about 6.1, about 6.2, about 6.3, about 6.4, about 6.5, or any range including and/or in between any two of these values. The pH of the first solvent and the second solvent after the adjusting step for each and of the third solvent may independently be for each solvent about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, or any range including and/or in between any two of these values.

The phage, first LDB, and second LDB may each independently be included in the first, second, and third solvent (respectively) at a concentration of about 1*10⁵ plaque forming units per milliliter solvent (PFU/mL) to about 1*10¹⁰ PFU/mL. For example, the phage may be included in the first solvent at a concentration of about 1*10⁸ PFU/mL. Accordingly, the phage, first LDB, and second LDB may each independently be included at a concentration of about 1*10⁵ PFU/mL, about 1*10⁶ PFU/mL, about 1*10⁷ PFU/mL, about 1*10⁸ PFU/mL, about 1*10⁹ PFU/mL, about 1*10¹⁰ PFU/mL, or any range including and/or in between any two of these values.

The first labelling moiety, second labelling moiety, and third labelling moiety may be included the first, second, and third solvent (respectively) at a concentration of about 1 nanomolar (nM) to about 10 millimolar (mM); thus, each labeling moiety may be included in their respective solvent at about 1 nM, about 10 nM, about 50 nM, about 100 nM, about 200 nM, about 300 nM, about 400 nM, about 500 nM, about 600 nM, about 700 nM, about 800 nM, about 900 nM, about 1 micromolar (μM), about 10 about 50 about 100 about 200 about 300 about 400 about 500 about 600 about 700 about 800 about 900 about 1 mM, about 2 mM, about 3 mM, about 4 mM, about 5 mM, about 6 mM, about 7 mM, about 8 mM, about 9 mM, about 10 mM, or any range including and/or in between any two of these values. The first activating agent, the second activating agent, the first carbodiimide, the second carbodiimide, and the activated linker may each independently be included at a concentration within the respective solvent of about 0.1 times to about 10 times the concentration of the respective labelling moiety, such as a concentration that is about 0.1 times, about 0.5 times, about 1 times (i.e., about equal to), about 2 times, about 3 times, about 4 times, about 5 times, about 7 times, about 8 times, about 9 times, or about 10 times (or any range including and/or in between any two of these values) the concentration of the respective labelling moiety.

In any embodiment of the method, a cosolvent in addition to water may be included in any one or more of the first, second, and third solvent. Any one or more of the first, second, and third solvent may include in addition to water an alcohol cosolvent (e.g., methanol (CH₃OH), ethanol (EtOH), isopropanol (iPrOH), trifluoroethanol (TFE), butanol (BuOH), ethylene glycol, propylene glycol), an ether cosolvent (e.g., tetrahydrofuran (THF), 2-methyltetrahydrofuran (2Me-THF), dimethoxyethane (DME), dioxane), an ester cosolvent (e.g., ethyl acetate, isopropyl acetate), a ketone cosolvent (e.g., acetone, methylethyl ketone, methyl isobutyl ketone), an amide cosolvent (e.g., dimethylformamide (DMF), dimethylacetamide (DMA)), a nitrile cosolvent (e.g., acetonitrile (CH₃CN), propionitrile (CH₃CH₂CN), benzonitrile (PhCN)), a sulfoxide cosolvent (e.g., dimethyl sulfoxide (DMSO)), a sulfone cosolvent (e.g., sulfolane), or a mixture of any two or more thereof. While specific cosolvents have been disclosed, numerous other solvents that would be known to those having ordinary skill in the art having the present disclosure before them are likewise contemplated for use. In any embodiment herein, the amount of cosolvent included based on weight of the first, second, or third solvent may be about 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, or any range including or in between any two of these values.

In any embodiment of the method, the first labelling moiety, the second labelling moiety (when present), and the third labelling moiety (when present) may independently be Atto Dye 590, Atto Dye 594, an amine- or carboxylate-functionalized quantum dot (e.g., amine-funcationalized Q-Dot 545, amine-funcationalized Q-Dot 585, amine-funcationalized Q-Dot 605, amine-funcationalized Q-Dot 655, amine-funcationalized Q-Dot 705, amine-funcationalized Q-Dot 800, carboxylate-funcationalized Q-Dot 545, carboxylate-funcationalized Q-Dot 585, carboxylate-funcationalized Q-Dot 605, carboxylate-funcationalized Q-Dot 655, carboxylate-funcationalized Q-Dot 705, carboxylate-funcationalized Q-Dot 800), fluorescein isothiocyanate (FITC), 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); or VIC®.

The first activating agent, the second activating agent (when present), and the third activating agent (when present) may each independently be N-hydroxysuccinimide (NHS), N-hydroxysulfosuccinimide (Sulfo-NHS), hydroxybenzotriazole (HOBt), 1-hydroxy-7-azabenzotriazole (HOAt), pentafluorophenol, or a combination of any two or more thereof.

The first carbodiimmide and the second carbodiimmide (when present) may each independently be N,N′-dicyclohexylcarbodiimide (DCC), 1-ethyl-3-(3-dimethylaminopropyl)carbodiimide (EDC), N,N′-diisopropylcarbodiimide (DIC), 1-[3-(dimethylamino)propyl]-3-ethylcarbodiimide methiodide (EDC-MeI), N,N′-di-tert-butylcarbodiimide, N-cyclohexyl-N′-(2-morpholinoethyl)carbodiimide methyl-p-toluenesulfonate, or a combination of any two or more thereof.

In a related aspect, the present disclosure provides a method for generating a LDB where the amide group or groups of the LDB include

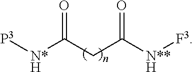

The method includes contacting a phage that includes an amine group disposed on the exterior surface of a capsid of the phage with a first labelling moiety comprising an amine group and an activated linker of the formula

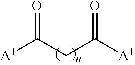

in the presence of a first solvent (that includes water) to provide a first LDB, where A¹ is a heteroatom of a first activating agent. Labelling moieties and activating agents are described previously in this disclosure. A pH of the first solvent may be about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, or any range including and/or in between any two of these values. A cosolvent in addition to water may be included in the first solvent, where such cosolvents and amounts are described earlier in this disclosure.

The phage may be included at a concentration of about 1*10⁵ plaque forming units per milliliter first solvent (PFU/mL) to about 1*10¹⁰ PFU/mL; the phage may be included at a concentration of about 1*10⁵ PFU/mL, about 1*10⁶ PFU/mL, about 1*10⁷ PFU/mL, about 1*10⁸ PFU/mL, about 1*10⁹ PFU/mL, about 1*10¹⁰ PFU/mL, or any range including and/or in between any two of these values.

The first labelling moiety may be included in the first solvent at a concentration of about 1 nM, about 10 nM, about 50 nM, about 100 nM, about 200 nM, about 300 nM, about 400 nM, about 500 nM, about 600 nM, about 700 nM, about 800 nM, about 900 nM, about 1 μM, about 10 μM, about 50 μM, about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, or any range including and/or in between any two of these values. The activated linker may be included within the first solvent at a concentration of about 0.1 times, about 0.5 times, about 1 times (i.e., about equal), about 2 times, about 3 times, about 4 times, about 5 times, about 7 times, about 8 times, about 9 times, about 10 times, or any range including and/or in between any two of these values, the concentration of the first labelling moiety.

Inclusion of additional labeling moieties may be achieved via similar steps as recited for any embodiment of the method of the previous aspect.

Additionally or alternatively, in any aspect herein, one or more reporter molecules may be intercalated into the nucleic acids of the bacteriophage of the present technology. Nucleic-acid reporters can fluoresce or absorb light, and localize to the specific bacterial host cell during infection, thus allowing detection of the target bacterial host cells via microscopy, luminometry, or flow cytometry. Examples of nucleic acid intercalating dyes include SYBR Gold, SYBR Green I, SYBR Safe, Quant-iT PicoGreen, Blue-Fluorescent SYTO dyes (e.g., SYTO 40, SYTO 41, SYTO 42, SYTO 45), Green-Fluorescent SYTO dyes (e.g., SYTO 9, SYTO 10, SYTO BC, SYTO 13, SYTO 16, SYTO 24, SYTO 21, SYTO 12, SYTO 11, SYTO 14, SYTO 25), Orange-Fluorescent SYTO dyes (e.g., SYTO 81, SYTO 80, SYTO 82, SYTO 83, SYTO 84, SYTO 85), Red-Fluorescent SYTO dyes (e.g., SYTO 64, SYTO 61, SYTO 17, SYTO 59, SYTO 62, SYTO 60, SYTO 63), cyanine dimer dyes (e.g., TOTO-1, YOYO-1, POPO-1, LOLO-1, BOBO-1, JOJO-1, POPO-3, BOBO-3, YOYO-3, TOTO-3), and other cyanine dyes, Acridine homodimer, Acridine orange, 7-AAD, ACMA, DAPI, Dihydroethidium, ethidium bromide, EthD-1, EthD-2, Hoechst 33258, Hoechst 33342, Hoechst 34580, hydroxystilbamidine, LDS 751, and the like.

Different combinations of fluorophore reporter molecules with different excitation and emission spectra can be added to both the exterior capsid and to the internal nucleic acids of the bacteriophage, permitting detection and discrimination of multiple bacterial species within a sample. In certain embodiments, bacteriophage comprising chemical- or fluorescently-labelled protein coats and/or nucleic acids may be subjected to phage genome engineering to produce a recombinant phage comprising a heterologous nucleic acid encoding one or more reporter proteins. In any embodiment herein, the heterologous nucleic acid may comprise an open reading frame that encodes a bioluminescent protein, a fluorescent protein, a chemiluminescent protein, an enzymatic protein, an affinity tag domain, or any combination thereof. In some embodiments, the encoded gene product(s) produces a detectable signal upon exposure to the appropriate stimuli, and the resulting signal permits detection of bacterial host cells infected by the recombinant phage. In certain embodiments, the open reading frame encodes a protein that serves as a marker that can be identified by screening bacterial host cells infected by a recombinant phage comprising a heterologous nucleic acid sequence comprising the open reading frame. Examples of such markers include by way of example and without limitation: a fluorescent label, a luminescent label, a chemiluminescence label, or an enzymatic label. In some embodiments, the heterologous nucleic acid sequence further comprises sequences naturally found in the bacteriophage, but placed at a non-normally occurring location in the genome.

In some embodiments, the length of the heterologous nucleic acid sequence is at least 100 bases, at least 200 bases, at least 300 bases, at least 400 bases, at least 500 bases, at least 600 bases, at least 700 bases, at least 800 bases, at least 900 bases, at least 1 kilobase (kb), at least 1.1 kb, at least 1.2 kb, at least 1.3 kb, at least 1.4 kb, at least 1.5 kb, at least 1.6 kb, at least 1.7 kb, at least 1.8 kb, at least 1.9 kb, at least 2.0 kb, at least 2.1 kb, at least 2.2 kb, at least 2.3 kb, at least 2.4 kb, at least 2.5 kb, at least 2.6 kb, at least 2.7 kb, at least 2.8 kb, at least 2.9 kb, at least 3.0 kb, at least 3.1 kb, at least 3.2 kb, at least 3.3 kb, at least 3.4 kb, at least 3.5 kb, at least 3.6 kb, at least 3.7 kb, at least 3.8 kb, at least 3.9 kb, at least 4.0 kb, at least 4.5 kb, at least 5.0 kb, at least 5.5 kb, at least 6.0 kb, at least 6.5 kb, at least 7.0 kb, at least 7.5 kb, at least 8.0 kb, at least 8.5 kb, at least 9.0 kb, at least 9.5 kb, at least 10 kb, or more. In certain embodiments, the heterologous nucleic acid sequence comprises a length that is less than or equal to a length selected from the group consisting of 1 kb, 2 kb, 3 kb, 4 kb, 5 kb, 6 kb, 7 kb, 8 kb, 9 kb, and 10 kb. In some embodiments, the heterologous nucleic acid sequence comprises a length that is less than or equal to the maximum length of heterologous nucleic acid sequence that can be packaged into a phage particle comprising the phage genome.

In some embodiments, the length of the heterologous nucleic acid sequence is from 100 to 500 bases, from 200 to 1,000 bases, from 500 to 1,000 bases, from 500 to 1,500 bases, from 1 kb to 2 kb, from 1.5 kb to 2.5 kb, from 2.0 kb to 3.0 kb, from 2.5 kb to 3.5 kb, from 3.0 kb to 4.0 kb, from 3.5 kb to 4.5 kb, from 4.0 kb to 5.0 kb, from 4.5 kb to 5.5 kb, from 5.0 kb to 6.0 kb, from 5.5 kb to 6.5 kb, from 6.0 kb to 7.0 kb, from 6.5 kb to 7.5 kb, from 7.0 kb to 8.0 kb, from 7.5 kb to 8.5 kb, from 8.0 kb to 9.0 kb, from 8.5 kb to 9.5 kb, or from 9.0 kb to 10.0 kb.

In some embodiments, the heterologous nucleic acid sequence is inserted into the phage genome with no loss of endogenous phage genomic sequence. In some embodiments, the heterologous nucleic acid sequence replaces an endogenous phage genomic sequence. In some embodiments, the heterologous nucleic acid sequence includes an endogenous phage genomic sequence that was previously excised from the phage genome. In certain embodiments, the heterologous nucleic acid sequence replaces an endogenous phage genomic sequence that is less than the length of the heterologous nucleic acid sequence. Accordingly, in some embodiments, the length of the recombinant phage genome is longer than the length of the wild-type phage genome. In some embodiments, the heterologous nucleic acid sequence replaces an endogenous phage genomic sequence that is greater than the length of the heterologous nucleic acid sequence. Thus, in some embodiments, the length of the recombinant phage genome is shorter than the length of the wild-type phage genome. In certain embodiments, the heterologous nucleic acid sequence replaces an endogenous phage genomic sequence that is equal to the length of the heterologous nucleic acid sequence.

In certain embodiments, the open reading frame of the heterologous nucleic acid encodes a protein that confers a phenotype of interest on a host cell infected by a recombinant phage expressing the heterologous nucleic acid. In some embodiments, the phenotype of interest is the expression of the gene product encoded by the open reading frame of the heterologous nucleic acid.

In certain embodiments, the open reading frame of the heterologous nucleic acid is operably linked to an expression control sequence that is capable of directing expression of the open reading frame, wherein the open reading frame encodes a bioluminescent protein, a fluorescent protein, a chemiluminescent protein, an enzymatic protein, an affinity tag domain, or any combination thereof. In some embodiments, the expression control sequence is located within the heterologous nucleic acid sequence. In other embodiments, the expression control sequence is located in the endogenous phage genome sequence. For example, the open reading frame may be inserted into the phage genome downstream of or in the place of an endogenous phage open reading frame sequence. In some embodiments, the expression control sequence is an inducible promoter or a constitutive promoter. See e.g., Djordjevic & Klaenhammer, Methods in Cell Science 20(1):119-126 (1998). The inducible promoter or constitutive promoter may be an endogenous phage promoter sequence, a non-endogenous phage promoter sequence, or a bacterial host promoter sequence. Additionally or alternatively, in some embodiments, the inducible promoter is a pH-sensitive promoter, or a temperature sensitive promoter. In some embodiments, the heterologous nucleic acid sequence comprises a first open reading frame and at least one supplemental open reading frame. In certain embodiments, the first and the at least one supplemental open reading frames are operably linked to the same expression control sequences. In some embodiments, the first and the at least one supplemental open reading frames are operably linked to different expression control sequences.

Fluorescent proteins include but are not limited to blue/UV fluorescent proteins (for example, TagBFP, Azurite, EBFP2, mKalamal, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, dsRed, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatable fluorescent proteins (for example, PA-GFP, PAmCherry1, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), fluorescein, rhodamine, and photoswitchable fluorescent proteins (for example, Dronpa).

Examples of bioluminescent proteins are aequorin (derived from the jellyfish Aequorea victoria) and luciferases (including luciferases derived from firefly and Renilla, nanoluciferase, red luciferase, luxAB, and the like). These proteins have also been genetically separated into two distinct functional domains that will generate light only when the protein domains are closely co-localized. A variety of emission spectrum-shifted mutant derivatives of both of these proteins have been generated over the past decade and have been used for multi-color imaging and co-localization within a living cell.

Examples of chemiluminescent protein include β-galactosidase, horseradish peroxidase (HRP), and alkaline phosphatase. Peroxidases generate peroxide that oxidizes luminol in a reaction that generates light, whereas alkaline phosphatases remove a phosphate from a substrate molecule, destabilizing it and initiating a cascade that results in the emission of light.

In some embodiments, the open reading frame of the heterologous nucleic acid comprises an epitope that can be detected with an antibody or other binding molecule. For example, an antibody that recognizes the epitope may be directly linked to a signal generating moiety (such as by covalent attachment of a chemiluminescent or fluorescent protein), or can be detected using at least one additional binding reagent such as a secondary antibody, directly linked to a signal generating moiety. In some embodiments, the epitope is absent in wild-type bacteriophage and the bacterial host cell. Accordingly, detection of the epitope in a sample demonstrates the presence of a bacterial host cell infected by a recombinant phage comprising a heterologous nucleic acid, wherein the open reading frame of the heterologous nucleic acid comprises the epitope.

In other embodiments, the open reading frame of the heterologous nucleic acid comprises a polypeptide affinity tag sequence, such that the expression product of the open reading frame comprises the tag fused to a polypeptide or protein encoded by the open reading frame (e.g., poly-histidine, FLAG, Glutathione S-transferase (GST) etc.).

In some embodiments, the open reading frame of the heterologous nucleic acid sequence comprises a biotin binding protein such as avidin, streptavidin, or neutrAvidin that can be detected with a biotin molecule conjugated to an enzyme (e.g., β-galactosidase, horseradish peroxidase (HRP), and alkaline phosphatase) or an antibody. In some embodiments, the antibody conjugated to a biotin molecule may be directly linked to a signal generating moiety (such as by covalent attachment of a chemiluminescent or fluorescent protein), or can be detected using at least one additional binding reagent such as a secondary antibody, directly linked to a signal generating moiety.

### Detection and Antibiotic Susceptibility Profiling Methods of the Present Technology

Accurate identification of bacterial species within a biological sample informs the selection of suitable therapies for treating bacterial infections. Labelled detector bacteriophages disclosed herein may be used to identify bacteria present in a biological sample (e.g., whole blood, plasma, serum) obtained from a subject. Such methods entail contacting the biological sample with an effective amount of a labelled detector bacteriophage disclosed herein, and detecting the presence of bacterial host cells infected by the labelled detector phage, wherein the labelled detector phage comprises at least one reporter molecule that (a) is present on the exterior surface of the phage capsid, (b) is present within the nucleic acids of the phage, and/or (c) is encoded by a heterologous nucleic acid located within the phage genome, thereby leading to the identification of at least one bacterial strain or species in the biological sample.

Additionally or alternatively, the labelled detector bacteriophages disclosed herein, may be used in methods for profiling antibiotic susceptibility of bacteria present in a biological sample (e.g., whole blood, plasma, serum). These methods include (a) contacting the biological sample with an antibiotic and an effective amount of a labelled detector bacteriophage disclosed herein, (b) detecting the presence of bacterial host cells infected by the labelled detector phage, wherein the labelled detector phage comprises at least one reporter molecule that (i) is present on the exterior surface of the phage capsid, (ii) is present within the nucleic acids of the phage, and/or (iii) is encoded by a heterologous nucleic acid located within the phage genome, and (c) determining that the antibiotic is effective in inhibiting the bacteria present in the biological sample when the number of labelled detector phage-infected bacterial host cells is reduced relative to that observed in an untreated control sample.

In some embodiments, identification of at least one bacterial strain or species includes detecting the signal of the one or more reporter molecules of the one or more labelled detector bacteriophages, e.g., detection of green fluorescence indicates the presence of bacterial species A whereas detection of blue fluorescence indicates the presence of bacterial species B. In some embodiments, the absence of at least one bacterial strain or species is identified by the lack of detectable signal of the one or more reporter molecules of the one or more labelled detector bacteriophages, e.g., undetectable expression of green fluorescence indicates the lack of bacterial species A in a test sample.

In some embodiments, the one or more labelled detector bacteriophages infect a single species of bacteria. In certain embodiments, the one or more labelled detector bacteriophage infect two or more species of bacteria. By way of example, but not by way of limitation, in some embodiments, the species of bacteria that are infected include Pseudomonas aeruginosa, Escherichia coli, Staphylococcus aureus, Klebsiella pneumoniae, Yersinia pestis, Bacillus anthracis, Burkholderia mallei, and Franciscella tularensis.

In some embodiments, the one or more labelled detector bacteriophages that infect two or more species of bacteria comprise different reporter molecules, wherein the labelled detector bacteriophages that infect the same species of bacteria comprise the same reporter molecule(s).

In some embodiments, detection of the reporter molecule signal is detection of the reporter molecule itself, e.g., a fluorescent protein. In some embodiments, detection of the reporter molecule signal is detection of an enzymatic reaction requiring the activity of the reporter molecule, e.g., expression of luciferase to catalyze luciferin to produce light.

In some embodiments, the signal of the one or more reporter molecules is detected in about 5, 10, 15, 20, 25, 30, 35, 40, 45, 50, 55, 60, 65, 70, 75, 80, 85, 90 minutes or any time between any two of the preceding values after contacting a sample with the one or more labelled detector bacteriophages disclosed herein.

In another aspect, the present disclosure provides a method for determining the antibiotic susceptibility of a bacterial strain or species in a test sample obtained from a subject comprising (a) contacting a plurality of test samples comprising bacterial cells with an effective amount of a labelled detector bacteriophage of the present technology and at least one antibiotic, wherein the plurality of test samples is derived from the subject and wherein the labelled detector phage comprises at least one reporter molecule that (i) is present on the exterior surface of the phage capsid, (ii) is present within the nucleic acids of the phage, and/or (iii) is encoded by a heterologous nucleic acid located within the phage genome, (b) detecting the signal of the reporter molecule of the labelled detector phage-infected bacterial cells in the plurality of test samples; and (c) determining that an antibiotic is effective in inhibiting the bacterial strain or species when the number of labelled detector phage-infected bacterial cells in the test sample is reduced relative to that observed in an untreated control sample comprising bacterial cells, wherein the untreated control sample is derived from the subject.

In other embodiments, the method further comprises determining that the bacterial strain or species in the test sample is resistant to an antibiotic when the number of labelled detector phage-infected bacterial cells in the antibiotic treated test sample is comparable to that observed in the untreated control sample. In certain embodiments, the method for determining the antibiotic susceptibility of a bacterial strain or species in a test sample does not require the culturing of bacterial cells from a test sample.

In some embodiments, the at least one antibiotic is one or more of rifampicin, tetracycline, levofloxacin, and ampicillin. Examples of other antibiotics include penicillin G, methicillin, oxacillin, amoxicillin, cefadroxil, ceforanid, cefotaxime, ceftriaxone, doxycycline, minocycline, amikacin, gentamycin, kanamycin, neomycin, streptomycin, tobramycin, azithromycin, clarithromycin, erythromycin, ciprofloxacin, lomefloxacin, norfloxacin, chloramphenicol, clindamycin, cycloserine, isoniazid, rifampin, teicoplanin, quinupristin/dalfopristin, linezolid, pristinamycin, ceftobiprole, ceftaroline, dalbavancin, daptomycin, mupirocin, oritavancin, tedizolid, telavancin, tigecycline, ceftazidime, cefepime, piperacillin, ticarcillin, virginiamycin, netilmicin, paromomycin, spectinomycin, geldanamycin, herbimycin, rifaximin, loracarbef, ertapenem, doripenem, imipenem/cilastatin, meropenem, cefazolin, cefalotin, cephalexin, cefaclor, cefamandole, cefoxitin, cefprozil, cefuroxime, cefixime, cefdinir, cefditoren, cefoperazone, cefpodoxime, ceftibuten, ceftizoxime, lincomycin, dirithromycin, roxithromycin, troleandomycin, telithromycin, spiramycin, aztreonam, furazolidone, nitrofurantoin, posizolid, radezolid, torezolid, azlocillin, carbenicillin, cloxacillin, dicloxacillin, flucloxacillin, mezlocillin, nafcillin, penicillin V, temocillin, bacitracin, colistin, polymyxin B, enoxacin, gatifloxacin, gemifloxacin, moxifloxacin, nalidixic acid, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (Co-trimoxazole) (TMP-SMX), sulfonamidochrysoidine, demeclocycline, oxytetracycline, clofazimine, dapsone, capreomycin, ethambutol, ethionamide, pyrazinamide, rifabutin, rifapentine, arsphenamine, fosfomycin, fusidic acid, metronidazole, platensimycin, thiamphenicol, tinidazole, trimethoprim(Bs) and vancomycin.

In some embodiments of the method, the differences in the reporter molecule signal of the labelled detector bacteriophage observed in the antibiotic treated test sample and the untreated control sample is defined as μ.

Additionally or alternatively, in some embodiments of the method, the signal of the reporter molecule is detected in about 5, 10, 15, 20, 25, 30, 35, 40, 45, 50, 55, 60, 65, 70, 75, 80, 85, 90 minutes or any time between any two of the preceding values after contacting a test sample with a labelled detector bacteriophage disclosed herein.

In some embodiments, two or more test samples are tested for antibiotic susceptibility in series. In some embodiments, two or more test samples are tested for antibiotic susceptibility in parallel. In some embodiments, one or more test samples are tested for antibiotic susceptibility in a running assay (where resistance or sensitivity to one antibiotic is determined and the resistance or sensitivity to a second, third, fourth, fifth, etc., antibiotic is being assayed).

Additionally or alternatively, in some embodiments of the bacterial identification and/or antibiotic susceptibility profiling methods disclosed herein, the bacterial cells are isolated/purified from the test samples obtained from the subject. Purification steps may include incubating the test samples with distilled water to form a mixture, centrifuging the mixture to form a pellet that includes bacterial cells, and re-suspending the pellet to form a bacterial suspension comprising isolated bacterial cells after discarding the supernatant. The pellet may be re-suspended in a phosphate buffer. Alternatively, acoustophoresis may be used to separate larger components of blood from blood plasma and bacteria. In another embodiment, a microfluidic trap may be used to capture the bacteria for purification and concentration. Other methods of isolating bacterial cells include capturing the bacteria on a filter to remove plasma and smaller components, and resuspending the bacteria in a clean buffer. These purification methods are useful for purifying other types of biological samples, such as urine samples, swabs, or environmental samples.

In certain embodiments of the methods disclosed herein, mixing the test sample with distilled water will lead to the lysis of cells that lack cell walls (e.g., mammalian cells and red blood cells) while leaving cells with cell walls (e.g., bacteria) intact. Without wishing to be bound by theory, in some embodiments, the removal of cells that lack cell walls enhances the detection of reporter molecules in bacterial cells infected with a labelled detector bacteriophage, as intact non-bacterial cells (e.g., red blood cells) may quench the signal of the reporter molecules.

In some embodiments of the methods of the present technology, the mixture is about 90% distilled water and 10% test sample, about 80% distilled water and 20% test sample, about 70% distilled water and 30% test sample, about 60% distilled water and 40% test sample, about 50% distilled water and 50% test sample, about 40% distilled water and 60% test sample, about 30% distilled water and 70% test sample, about 20% distilled water and 80% sample, or about 10% distilled water and 90% test sample.

In some embodiments of the methods disclosed herein, the mixture is incubated for 1, 2, 3, 4, 5, 6, 7, 8, 9, 10, 11, 12, 13, 14, or 15 minutes, or any time between two of the previously listed time points. Additionally or alternatively, in certain embodiments of the methods disclosed herein, the mixture is centrifuged for 1, 2, 3, 4, 5, 6, 7, 8, 9, 10, 11, 12, 13, 14, or 15 minutes, or any time between two of the previously listed time points.

Additionally or alternatively, in certain embodiments of the methods disclosed herein, each of the one or more test samples comprise between about 10 to 20, about 5 to 500, about 10 to 400, about 20 to 300, about 30 to 300, about 40 to 200 or about 50 to 100 bacterial cells. In some embodiments of the methods disclosed herein, each of the one or more samples comprises between about less than 10, about 10 to 10,000, about 200 to 9,000, about 300 to 8,000, about 400 to 7,000, about 500 to 6,000, about 600 to 5,000, about 700 to 4,000, about 800 to 3,000, about 900 to 2,000, or about 1,000 to 1,500 bacterial cells.

In any of the above embodiments of the methods of the present technology, the test sample is blood, sputum, mucus, lavage, saliva, or a swab obtained from the subject.

In some embodiments of the methods disclosed herein, the test sample is obtained from a mammal, including, for example, farm animals, such as sheep, pigs, cows, and horses; pet animals, such as dogs and cats; and laboratory animals, such as rats, mice and rabbits. In one embodiment, the mammal is a human.

### Microwell Detection Methods

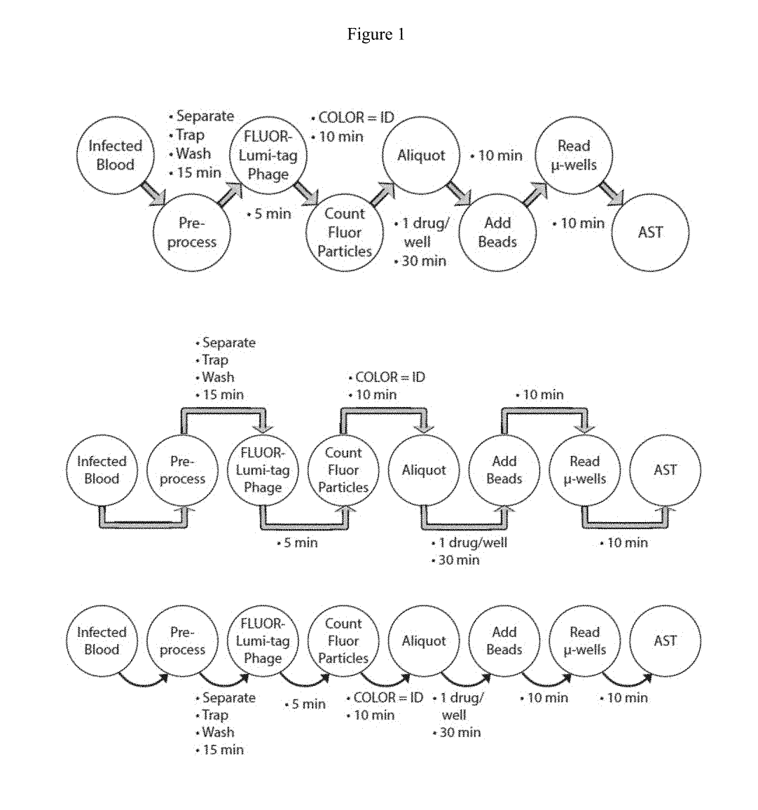

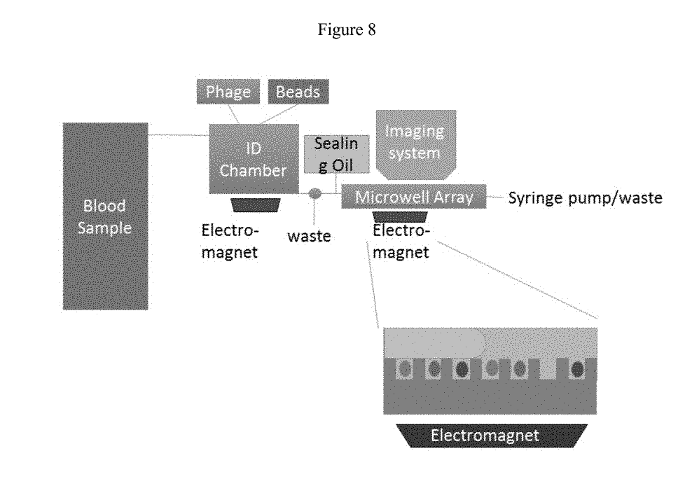

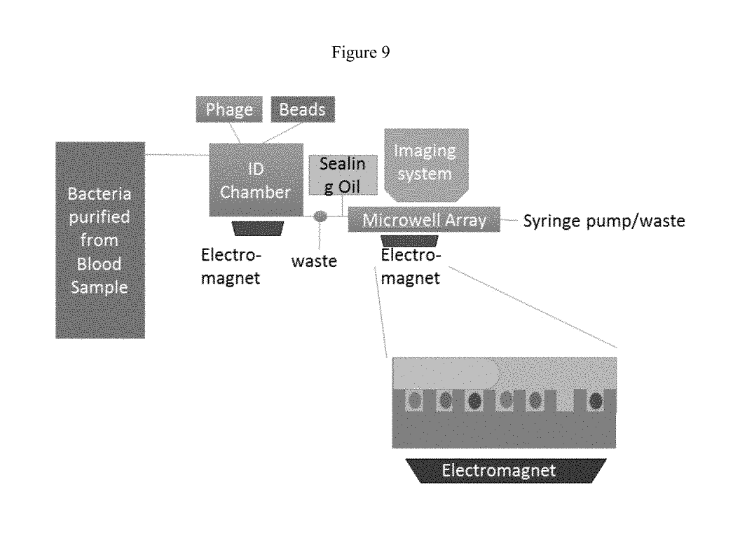

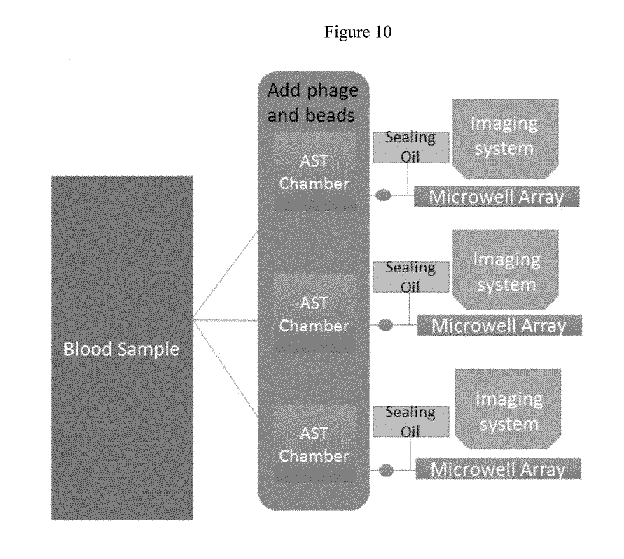

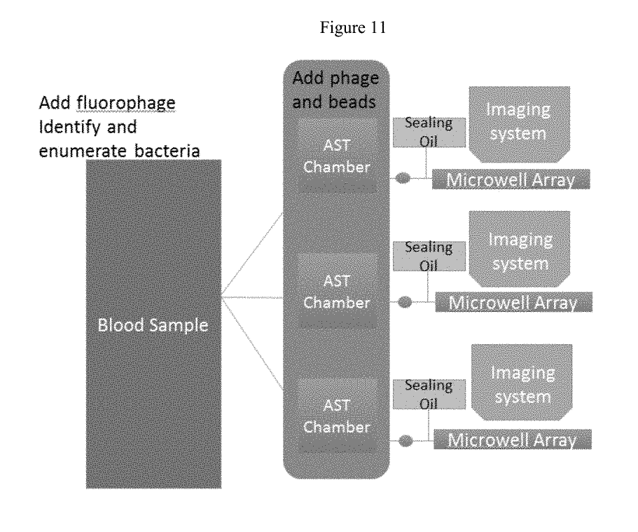

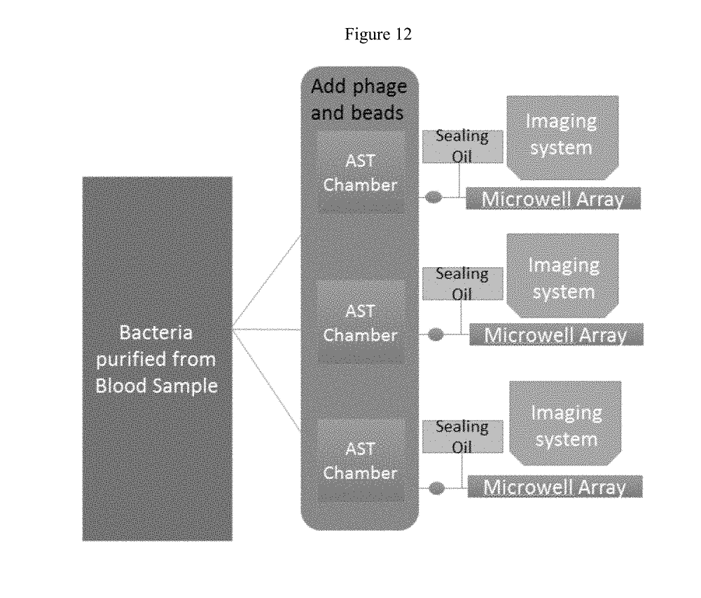

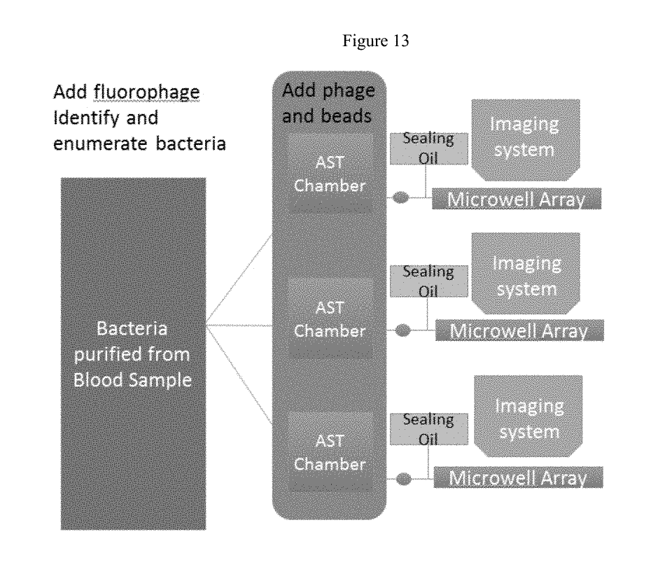

FIG. 1 is a schematic of four different embodiments of methods for identifying and antibiotic susceptibility profiling of bacteria using labelled detector bacteriophages of the present technology in a microwell assay format. FIGS. 8-13 show methods for bacterial detection, enumeration and antibiotic susceptibility profiling in a purified or unpurified sample using labelled detector bacteriophages of the present technology in microwells. In each embodiment, the biological sample is diluted in an infection buffer (e.g., TSB (Tryptic Soy Broth) supplemented with metal ions such as calcium or magnesium (typically 10 mM Mg, and 1 mM Ca) to aid in viral infection).

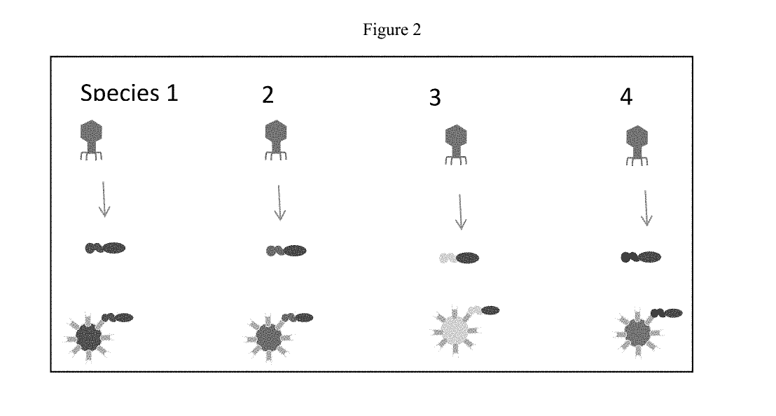

In certain embodiments, the labelled detector phage of the present technology comprises a heterologous nucleic acid encoding one or more reporter proteins. In some embodiments, the one or more reporter proteins comprise an active domain of any enzyme and an affinity tag domain (FIG. 2). For example, in some embodiments, the active domain of the enzyme is the alpha chain of beta galactosidase, and the affinity tag domain is streptavidin. These methods show superior sensitivity and specificity compared to conventional techniques for bacterial identification, bacterial enumeration, and measuring viability of bacterial cells in a biological sample.

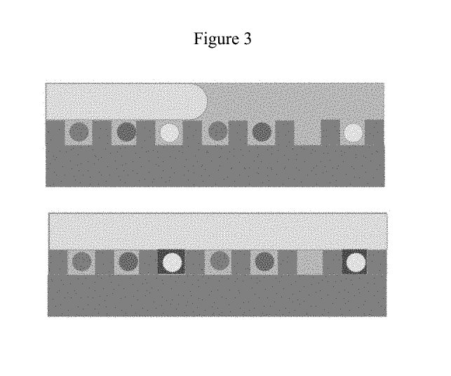

In one embodiment, the methods of the present technology comprise the use of microbeads or other solid support microstructures that are coated with reagents that allow capture and separation of (a) a bacterial host cell infected by a labelled detector phage disclosed herein, or (b) one or more reporter proteins expressed by a bacterial host cell infected by a labelled detector phage disclosed herein from the biological sample. See FIG. 3. In some embodiments, the one or more reporter proteins expressed by a labelled detector phage-infected bacterial host cell comprise a polypeptide affinity tag (e.g., poly-histidine, FLAG, Glutathione S-transferase (GST), a biotin binding protein etc.) fused to a bioluminescent protein, a fluorescent protein, a chemiluminescent protein, an enzymatic protein, or any combination thereof, and the microbeads or other solid support microstructures are coated with reagents that bind to the polypeptide affinity tag. The isolated labelled detector phage-infected bacterial host cells, or reporter proteins expressed by the labelled detector phage-infected bacterial host cells are then deposited in a microwell array, and optionally an enzymatic substrate. The wells may be sealed by mechanical sealing, oil sealing, or by another means. The captured labelled detector phage-infected bacterial host cells, or reporter proteins expressed by the labelled detector phage-infected bacterial host cells may be detected via microscopy, scanning, or other imaging assays. In some embodiments, a biological sample is infected with at least two labelled detector phages disclosed herein. Capture molecules useful in isolating bacterial cells include bacteriophages, or bacteriophage proteins, antibodies, complement system proteins such as mannose-binding-lectin, TLR extracellular domains, or other proteins that non-specifically bind to bacteria.

Additionally or alternatively, in certain embodiments of the methods disclosed herein, the bacterial cells in the sample are purified to remove contaminating mammalian cells and plasma proteins prior to contact with the labelled detector phage of the present technology. Purification steps include, but not limited to, blood cell removal, buffer exchange, and concentration of isolated bacterial cells. A number of different methods can be employed to remove the contaminating cells and plasma including mechanical filtration, acoustic separation, dielectrophoretic separation, optical trapping, and fluidic separation employing microfluidic devices.

Detection of the one or more reporter proteins indicates the occurrence of phage infection and the presence of viable bacterial cells in the biological sample. The signal intensity of the one or more reporter proteins are correlated with the number of isolated labelled detector phage-infected bacterial host cells, or reporter proteins expressed by the labelled detector phage-infected bacterial host cells captured on the microbeads or other solid support microstructures. The ratio of signal intensity above noise is used to determine whether a particular bacterial strain is affected by a particular antibiotic.

In order to properly assess antibiotic susceptibility, it is important that bacteria are equally distributed into the different microwells containing antibiotics. If bacteria are not equally distributed, then a decrease in signal of the labelled detector bacteriophage could be due to either lower counts of bacteria within a well or due to true antibiotic susceptibility. In some embodiments, the number of bacteria within a sample is expanded through conventional growth conditions.

In certain embodiments of the methods disclosed herein, the bacterial cells in a sample are individually enumerated using any labelled detector bacteriophage of the present technology. For example, the labelled detector bacteriophage may comprise a reporter molecule that is attached to the exterior surface of the bacteriophage capsid or intercalated within the nucleic acids of the bacteriophage. Enumeration may be carried out using an optical system. Examples of optical systems include an imaging or single-point flow-based detection system, such as a microscope or flow cytometer, respectively. The signal intensity of the labelled detector bacteriophage is directly related to the number of viable bacteria. In this scenario, the total number of bacterial cells in a sample can be normalized to the number of bacterial cells that are coated with the labelled detector bacteriophage of the present technology. This normalization corrects for samples or aliquots where there are fewer bacterial cells within a biological sample and helps reduce the frequency of false negative results. Additionally or alternatively, in certain embodiments of the methods disclosed herein, the bacterial cells in the sample are purified to remove contaminating mammalian cells and plasma proteins prior to contact with the labelled detector phage of the present technology. Purification steps include, but not limited to, blood cell removal, buffer exchange, and concentration of isolated bacterial cells. A number of different methods can be employed to remove the contaminating cells and plasma including mechanical filtration, acoustic separation, dielectrophoretic separation, optical trapping, and fluidic separation employing microfluidic devices.

Accordingly, in one aspect, the present technology provides a method for identifying at least one bacterial strain or species in a biological sample obtained from a subject comprising contacting the biological sample with an effective amount of a labelled detector bacteriophage disclosed herein, and detecting the presence of bacterial cells infected by the labelled detector phage, wherein the labelled detector phage comprises at least one reporter molecule that (a) is present on the exterior surface of the phage capsid, (b) is present within the nucleic acids of the phage, and/or (c) is encoded by a heterologous nucleic acid located within the phage genome, thereby leading to the identification of at least one bacterial strain or species in the biological sample.

In another aspect, the present technology provides a method for determining the antibiotic susceptibility of a bacterial strain or species in a biological sample obtained from a subject comprising (a) contacting a plurality of test samples comprising bacterial cells with an effective amount of a labelled detector bacteriophage of the present technology and at least one antibiotic, wherein the plurality of test samples is derived from the subject and wherein the labelled detector phage comprises at least one reporter molecule that (i) is present on the exterior surface of the phage capsid, (ii) is present within the nucleic acids of the phage, and/or (iii) is encoded by a heterologous nucleic acid located within the phage genome, (b) detecting the signal of the at least one reporter molecule of the labelled detector phage-infected bacterial cells in the plurality of test samples; and (c) determining that an antibiotic is effective in inhibiting the bacterial strain or species when the number of labelled detector phage-infected bacterial cells is reduced relative to that observed in an untreated control sample comprising bacterial cells, wherein the untreated control sample is derived from the subject. The at least one reporter molecule may be a fluorescent label, a luminescent label, a colorimetric label, an electrochemical label (e.g., enzymes that create an electrochemical signal such as HRP, LDH etc., or catalysts such as gold or platinum nanoparticles that can generate an electrochemical signal), or a mechanical label (e.g., microbeads that are mechanically detectable) as described herein. The methods of the present technology may be carried out in a microwell format and are useful in enumerating the bacterial strain or species present in the biological sample. Careful enumeration of bacterial cells provides a reference for comparing numerical values for bacterial cell physiology in the presence or absence of antibiotics. The methods disclosed herein are an improvement over current methods that rely on equal segregation of bacterial cells into separate drug testing chambers.

Examples of suitable fluorescent labels working individually or in combination: Atto Dye 590, Atto Dye 594, an amine- or carboxylate-functionalized quantum dot (e.g., amine-funcationalized Q-Dot 545, amine-funcationalized Q-Dot 585, amine-funcationalized Q-Dot 605, amine-funcationalized Q-Dot 655, amine-funcationalized Q-Dot 705, amine-funcationalized Q-Dot 800, carboxylate-funcationalized Q-Dot 545, carboxylate-funcationalized Q-Dot 585, carboxylate-funcationalized Q-Dot 605, carboxylate-funcationalized Q-Dot 655, carboxylate-funcationalized Q-Dot 705, carboxylate-funcationalized Q-Dot 800), fluorescein isothiocyanate (FITC), 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®.

Other suitable labels include nucleic acid intercalating dyes such as SYBR Gold, SYBR Green I, SYBR Safe, Quant-iT PicoGreen, Blue-Fluorescent SYTO dyes (e.g., SYTO 40, SYTO 41, SYTO 42, SYTO 45), Green-Fluorescent SYTO dyes (e.g., SYTO 9, SYTO 10, SYTO BC, SYTO 13, SYTO 16, SYTO 24, SYTO 21, SYTO 12, SYTO 11, SYTO 14, SYTO 25), Orange-Fluorescent SYTO dyes (e.g., SYTO 81, SYTO 80, SYTO 82, SYTO 83, SYTO 84, SYTO 85), Red-Fluorescent SYTO dyes (e.g., SYTO 64, SYTO 61, SYTO 17, SYTO 59, SYTO 62, SYTO 60, SYTO 63), cyanine dimer dyes (e.g., TOTO-1, YOYO-1, POPO-1, LOLO-1, BOBO-1, JOJO-1, POPO-3, BOBO-3, YOYO-3, TOTO-3), and other cyanine dyes, Acridine homodimer, Acridine orange, 7-AAD, ACMA, DAPI, Dihydroethidium, ethidium bromide, EthD-1, EthD-2, Hoechst 33258, Hoechst 33342, Hoechst 34580, hydroxystilbamidine, LDS 751, and the like.

Additionally or alternatively, in some embodiments of the methods disclosed herein, the signal of the at least one reporter molecule is measured in about 5, 10, 15, 20, 25, 30, 35, 40, 45, 50, 55, 60, 65, 70, 75, 80, 85, 90 minutes or any time between any two of the preceding values after contacting a biological sample with a labelled detector bacteriophage disclosed herein.

In some embodiments of the methods disclosed herein, the antibiotic susceptibility of the bacterial strain or species can be determined in no more than 15 minutes after contacting a biological sample with a labelled detector bacteriophage disclosed herein and an antibiotic. Various concentrations of antibiotics may be employed in these microwell assays.

In any of the above embodiments, the methods of the present technology further comprise the use of a fluidics assembly, wherein sample preparation, bacterial purification, reagent mixing, thermal incubation, washing, substrate addition, microwell-sealing, and optical detection can be accomplished in an integrated fashion.

Additionally or alternatively, in some embodiments of the methods of the present technology, the at least one reporter molecule may be captured on microbeads or other solid support microstructures. In certain embodiments, the at least one reporter molecule comprises an affinity domain that specifically binds to the microbeads or solid support microstructures. The microbeads or solid support microstructures may be coded to facilitate the identification of a specific bacteria strain or species in the sample. Microbeads or solid support microstructures may be encoded by color, fluorescence, magnetism, shape, size, absorbance, or other distinguishable physical features that are useful to facilitate the identification of the bacteria in the sample. The microbeads or other solid support microstructures may optionally be coated with reagents that allow capture and separation of (a) a bacterial host cell infected by a labelled detector phage disclosed herein, or (b) at least one reporter molecule expressed by a bacterial host cell infected by a labelled detector phage disclosed herein from the biological sample.

The at least one reporter molecule may be directly detected or indirectly detected through the use of a secondary reagent. Additionally or alternatively, in some embodiments, the at least one reporter molecule promotes the association of another molecule (e.g., an enzyme) with the microbeads or solid support microstructures. In some embodiments of the methods disclosed herein, the presence of the at least one reporter molecule, and the identity of the coded microbeads or solid support microstructures are simultaneously determined.

In some embodiments of the methods disclosed herein, the microbeads or solid support microstructures are captured in a microwell array. For example, in some embodiments, the microwell array contains no more than 1 bead/well. In other embodiments, the microwell array contains more than 1 bead/well. The microwells may be sealed by mechanical sealing, oil sealing, or by another means.

In other embodiments of the methods disclosed herein, the microbeads or solid support microstructures are spread on a surface that does not contain microwells. The spread out microbeads can be assayed by optical imaging, or another means to detect the association of the at least one reporter molecule with the micro-beads.

Additionally or alternatively, the biological sample is infected with at least two labelled detector phages disclosed herein. In certain embodiments, the at least two labelled detector phages may be labelled with different chemical fluorophores, such that the multiple bacterial species infected by the at least two labelled detector phages can be readily discriminated (e.g., green fluorophore for Pseudomonas and blue fluorophore for E. coli). Additionally or alternatively, in some embodiments, the at least two labelled detector phages each have distinct host ranges. For example, the at least two labelled detector phages may each target distinct bacterial host species such as E. coli, Staphylococcus aureus, Pseudomonas, Acinetobacter baumannii, Enterococcus faecalis, etc.

In some embodiments of the methods disclosed herein, the biological sample comprises no more than 10 bacterial cells/ml, about 10 to 20 bacterial cells/ml, about 5 to 50 bacterial cells/ml, about 50 to 400 bacterial cells/ml, about 20 to 300 bacterial cells/ml, about 30 to 500 bacterial cells/ml, about 40 to 200 bacterial cells/ml, or about 50 to 450 bacterial cell s/ml.

In some embodiments of the methods disclosed herein, the biological sample comprises between about less than 10, about 10 to 10,000, about 200 to 9,000, about 300 to 8,000, about 400 to 7,000, about 500 to 6,000, about 600 to 5,000, about 700 to 4,000, about 800 to 3,000, about 900 to 2,000, or about 1,000 to 1,500 bacterial cells.

### Kits

The present technology provides kits for bacteria identification and antibiotic susceptibility profiling.

In one aspect, the kits of the present technology comprise one or more coded/labeled vials that contain a plurality of the labelled detector bacteriophages disclosed herein, and instructions for use.

In some embodiments, each coded/labeled vial containing a plurality of labelled detector bacteriophages corresponds to a different bacteriophage type. In other embodiments, each coded/labeled vial containing a plurality of labelled detector bacteriophages corresponds to the same bacteriophage type. In some embodiments, each phage vial is assigned a unique code that identifies the labelled detector bacteriophage in the phage vial, or the types of bacteria that the labelled detector bacteriophage strain infects. The unique code can be encoded by a machine discernible pattern, such as a bar code, a QR code, an alphanumeric string, or any other pattern that can be discerned by a reader. Each unique code may be shown as, for example, a bar code sticker on a vial or container storing a corresponding labelled detector phage sample. In some embodiments, the kit is stored under conditions that permit the preservation of the labelled detector bacteriophages for extended periods, such as under bacteriophage-specific, controlled temperature, moisture, and pH conditions. The kits may further comprise coded microbeads or other solid support microstructures for capture purposes. Microbeads or solid support microstructures may be encoded by color, fluorescence, magnetism, shape, size, absorbance, or other distinguishable physical features that are useful to facilitate the identification of the bacteria in the sample.

Additionally or alternatively, in some embodiments, the kits further comprise vials containing natural or non-natural bacterial host cells. In some embodiments, the bacterial host cells are E. coli. In certain embodiments, the bacterial host cells are E. coli strain DH10B.

The kits may also comprise software for automated analysis, containers, packages such as packaging intended for commercial sale and the like.

The kit may further comprise one or more of: wash buffers and/or reagents, hybridization buffers and/or reagents, labeling buffers and/or reagents, and detection means. The buffers and/or reagents are usually optimized for the particular detection technique for which the kit is intended. Protocols for using these buffers and reagents for performing different steps of the procedure may also be included in the kit.

Additionally or alternatively, the kits disclosed herein may also include coded and labeled vials that contain a plurality of antibiotics. In some embodiments, the plurality of antibiotics comprises one or more of rifampicin, tetracycline, levofloxacin, and ampicillin. Other examples of antibiotics include penicillin G, methicillin, oxacillin, amoxicillin, cefadroxil, ceforanid, cefotaxime, ceftriaxone, doxycycline, minocycline, amikacin, gentamycin, kanamycin, neomycin, streptomycin, tobramycin, azithromycin, clarithromycin, erythromycin, ciprofloxacin, lomefloxacin, norfloxacin, chloramphenicol, clindamycin, cycloserine, isoniazid, rifampin, teicoplanin, quinupristin/dalfopristin, linezolid, pristinamycin, ceftobiprole, ceftaroline, dalbavancin, daptomycin, mupirocin, oritavancin, tedizolid, telavancin, tigecycline, ceftazidime, cefepime, piperacillin, ticarcillin, virginiamycin, netilmicin, paromomycin, spectinomycin, geldanamycin, herbimycin, rifaximin, loracarbef, ertapenem, doripenem, imipenem/cilastatin, meropenem, cefazolin, cefalotin, cephalexin, cefaclor, cefamandole, cefoxitin, cefprozil, cefuroxime, cefixime, cefdinir, cefditoren, cefoperazone, cefpodoxime, ceftibuten, ceftizoxime, lincomycin, dirithromycin, roxithromycin, troleandomycin, telithromycin, spiramycin, aztreonam, furazolidone, nitrofurantoin, posizolid, radezolid, torezolid, azlocillin, carbenicillin, cloxacillin, dicloxacillin, flucloxacillin, mezlocillin, nafcillin, penicillin V, temocillin, bacitracin, colistin, polymyxin B, enoxacin, gatifloxacin, gemifloxacin, moxifloxacin, nalidixic acid, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (Co-trimoxazole) (TMP-SMX), sulfonamidochrysoidine, demeclocycline, oxytetracycline, clofazimine, dapsone, capreomycin, ethambutol, ethionamide, pyrazinamide, rifabutin, rifapentine, arsphenamine, fosfomycin, fusidic acid, metronidazole, platensimycin, thiamphenicol, tinidazole, trimethoprim(Bs) and vancomycin.

### EXAMPLES

### Example 1: Use of Labelled Detector Bacteriophages in Detecting and Identifying Bacteria

This Example demonstrates that the labelled detector bacteriophages of the present technology are useful in methods for identifying bacterial strains/species in a sample.

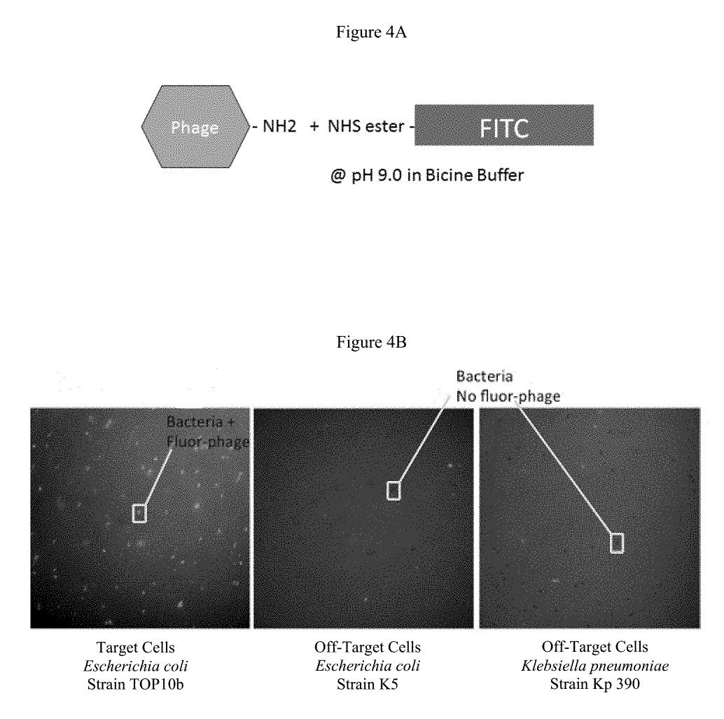

T7 bacteriophages were labelled with FITC via an alkaline-buffered condensation reaction of NHS-ester FITC and the primary amines (—NH₂) of lysine residues located at the surface of the T7 phage. See FIG. 4A. FITC labelled T7 bacteriophages were then purified from the unreacted dye by desalting column purification. In one example, the column was equilibrated in a final buffer, followed by application of the sample to the column. The sample was eluted by applying 1 fraction volume of final buffer and collecting 1 fraction at a time. Alternatively, the column was equilibrated in a final buffer and centrifuged, followed by application of the sample to the column and stacker. The column was centrifuged to collect the sample-containing flow-through that had been de-salted.

To ensure that FITC labelled T7 bacteriophages retained their original specificity, E. coli TOP10b cells (which are T7-specific host cells), K5 cells and Kp 390 cells were infected with FITC labelled T7 bacteriophages. The FITC labelled T7 phages were incubated with the bacterial cells at a MOI of 100 phage:1 bacterial cell. FIG. 4B shows that fluorescence was detected in the FITC labelled T7 infected E. coli TOP10b cells, whereas no fluorescence was detected in either the E. coli K5 cells or the K. pneumoniae strain Kp 390 cells.

These results demonstrate that the labelled detector bacteriophages of the present technology are useful in methods for identifying bacterial strains/species in a sample.

### Example 2: Methods for Generating Fluorophore Labelled Detector Bacteriophages

This Example demonstrates that the labelled detector bacteriophages of the present technology are useful in methods for identifying bacterial strains/species in a sample.

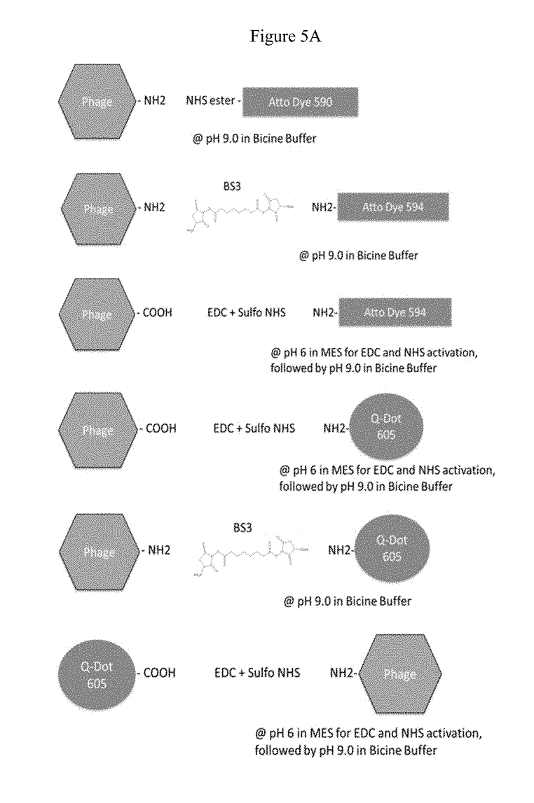

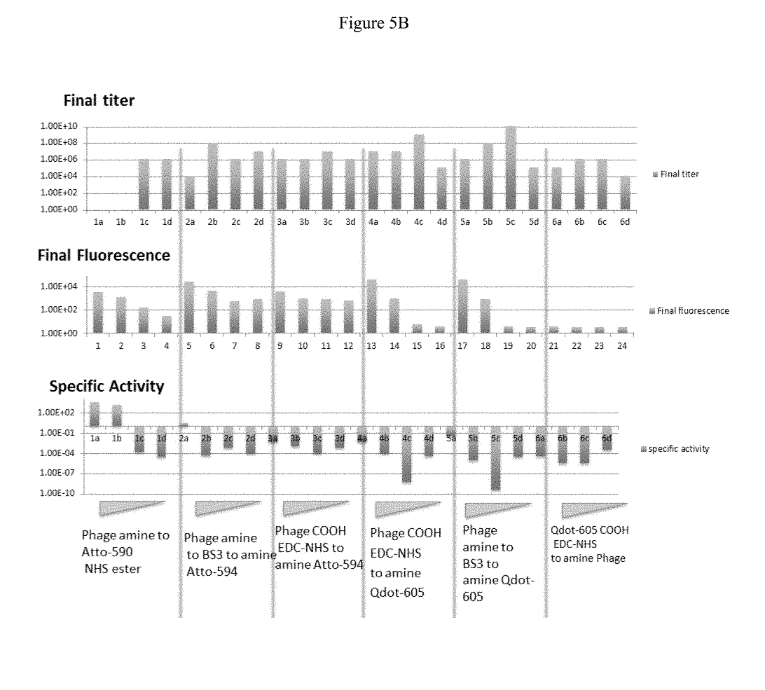

The relationship between the fluorescent intensity of the phage and the phage viability was evaluated by labelling phages under six different conditions with varying coupling chemistries, varying fluorophores, and varying fluorophore concentrations to determine the most effective coupling chemistries. See FIG. 5A and FIG. 5B. The phages were used at a concentration of 1*10⁶ PFU/mL; concentrations examined for the fluorophores (the ATTO dyes and Qdot 605 examples) were 2 μM to 2 mM. SulfoNHS, EDC, and BS3 concentrations were based on the concentration of fluorophore: for the ATTO dyes, SulfoNHS, EDC, and BS3 concentrations were 0.5 times, 0.25 times, and 0.25 times (respectively) the ATTO dye concentration employed; for the Qdot 605 examples, SulfoNHS, EDC, and BS3 concentrations were 5 times, 2.5 times, and 5 times (respectively) the particular Qdot 605 concentration employed.

FIG. 5A shows phages that were labelled with different fluorophores using varying coupling chemistries. The coupling chemistries were carried out for 12 hours (until completion) and the resulting phages were purified away from the unincorporated dye by dialysis for the smaller organic dyes, and by DEAE anion exchange chromatography for the Qdot labelled phages. FIG. 5B shows the results for final titer, final fluorescence, and total activity for each labelled phage described in FIG. 5A. For each labelled phage, the fluorophore concentration goes from the highest value to the lowest value (left to right). Total activity was calculated by multiplying the titer by the total fluorescence.

Results.

Phage-NH₂+BS3+NH₂-ATTO 594 and Phage-COOH+EDC+Sulfo NHS+NH₂-Qdot 605 exhibited high fluorescence levels and specific activity, even at high fluorophore concentrations. In contrast, Phage-NH₂+NHS ester-ATTO 590 exhibited lower specific activity levels at higher fluorophore concentrations, which is attributable to the low final titer, indicating lower phage viability at higher fluorophore concentrations. See FIG. 5B. Phage-NH₂+EDC+Sulfo NHS+COOH-Qdot 605 exhibited low levels of fluorescence across varying fluorophore concentrations, even with moderate final titer (See FIG. 5B), resulting in moderate total activity levels across varying fluorophore concentrations.

These results demonstrate that the labelled detector bacteriophages of the present technology are useful in methods for identifying bacterial strains/species in a sample.

### Example 3: Use of DNA Intercalating Dye-Labelled Detector Bacteriophage in Enumerating and Identifying Bacteria

This Example demonstrates the labelled detector bacteriophages of the present technology are useful in methods for identifying bacterial strains/species in a sample.

T7 bacteriophages were either stained with Hoechst dye (20 mM stock solution, Thermo Fisher Scientific, Cambridge, Mass.) or SYBR Gold dye (10,000× stock solution, Thermo Fisher Scientific, Cambridge, Mass.). Dye incorporation was carried out by contacting the phage with the dye overnight using Bicine buffer, a 10,000× dilution of the stock concentration of the respective dye.

To ensure that Hoechst labelled and SYBR Gold labelled T7 bacteriophages retained their original specificity, E. coli TOP10b cells and non-T7 specific bacterial host cells were incubated with the labelled T7 bacteriophages at a MOI of 100 phage:1 bacterial cell for 20 minutes.

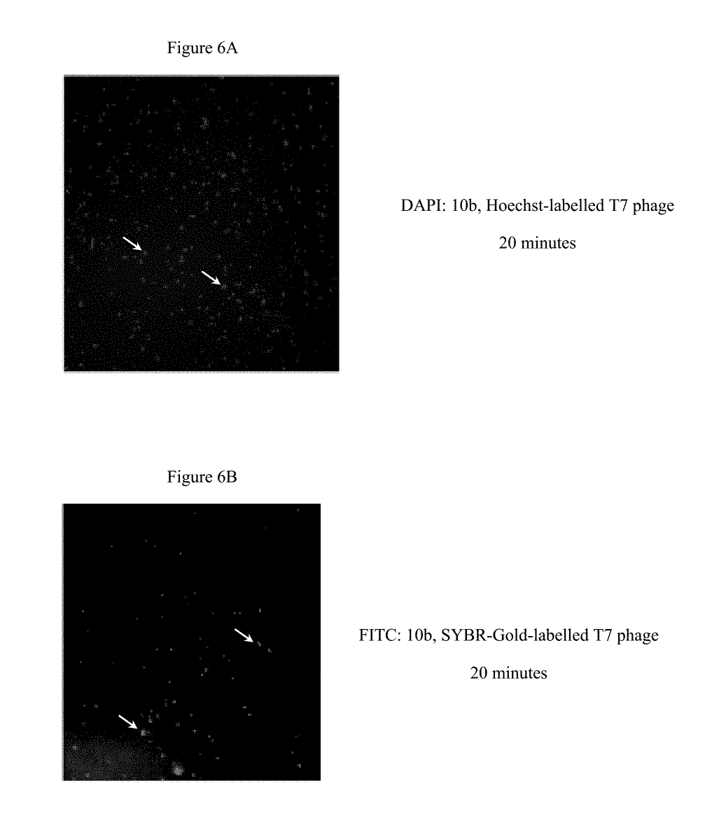

FIG. 6A and FIG. 6B show that fluorescence was detected in E. coli TOP10b cells infected with Hoechst labelled T7 bacteriophages and SYBR Gold labelled T7 bacteriophages respectively. No or background fluorescence was detected in Pseudomonas D12, an off-target bacterial host cell line. These results demonstrate that the Hoechst labelled and SYBR Gold labelled T7 bacteriophages specifically infected their normal host cells. These results demonstrate that labelled detector bacteriophages of the present technology are useful in methods for bacterial enumeration and identification.

The ability to perform multiplex bacterial identification and enumeration was evaluated using two different bacteriophage strains that target different host cells: PB1 phage and T7 phage.

PB1 bacteriophages were stained overnight with a 1000× dilution of SYBR Gold dye solution (10,000× stock solution, Thermo Fisher Scientific, Cambridge, Mass.). T7 bacteriophages were stained with Hoechst dye under the conditions described above. The SYBR Gold labelled PB1 and Hoechst labelled T7 bacteriophages were incubated for 20 minutes with a mixed culture of Pseudomonas aeruginosa (P. aeruginosa) strain A1 cells (which are the normal PB1 host cells) and E. coli TOP10b cells (which are the normal T7 host) at an MOI of 1000 phage:1 bacterial cell for each phage.

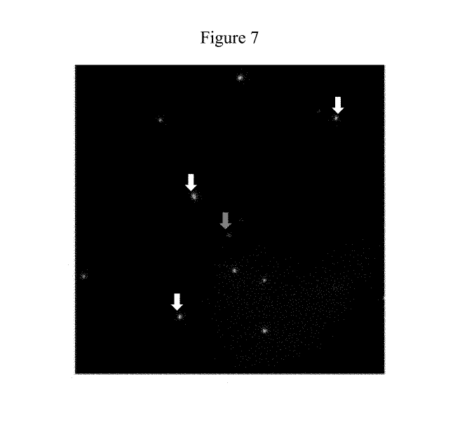

FIG. 7 shows the multispecies detection of E. coli TOP10b cells and P. aeruginosa strain A1 cells. Fluorescence was detected for E. coli TOP10b cells infected with Hoechst labelled T7 phages (marked with gray arrows). Fluorescence was also detected for P. aeruginosa strain A1 cells infected with SYBR Gold labelled PB1 phages (marked with white arrows).

These results demonstrate that the labelled detector bacteriophages of the present technology are useful in methods for identifying bacterial strains/species in a sample.

### Example 4: Use of Labelled Detector Bacteriophages in Antibiotic Susceptibility Profiling of Bacteria

This Example demonstrates that the labelled detector bacteriophages of the present technology are useful in methods for profiling the antibiotic susceptibility of bacterial strains/species.

FITC labelled T7 bacteriophages are generated and their specificity is confirmed following the protocols described in Example 1.

To test for antibiotic susceptibility, a sample of T7-specific bacterial host cells is aliquoted into several fractions. The FITC labelled T7 bacteriophages are incubated with each bacterial aliquot. One of the fractions is not treated with antibiotics and serves as a negative control. The other fractions are treated with a specific concentration of a particular antibiotic. The fluorescence of bacteria infected with the FITC labelled T7 bacteriophages is measured in microwells. The fluorescence of each antibiotic condition is compared to the negative control. The ratio of fluorescence signal intensity above noise is used to determine whether a particular bacterial strain is affected by the different antibiotics. The fluorescence will be attenuated if the bacteria are affected by the presence of the antibiotic.

These results will demonstrate that the labelled detector bacteriophages of the present technology are useful in methods for profiling the antibiotic susceptibility of bacterial strains/species.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A labelled detector bacteriophage (LDB) comprising
a capsid and nucleic acids wherein the capsid comprises an exterior surface relative to the nucleic acids of a phage; and
a labelling moiety, wherein the labelling moiety is covalently linked to the exterior surface of the phage via an amide group or groups.

2. The LDB of claim 1, wherein the LDB comprises two or more labelling moieties, wherein each labelling moiety is independently covalently linked to the exterior surface of the phage via an amide group or groups.

3. The LDB of claim 1, wherein the amide group or groups comprise 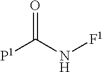
wherein P¹ represents the phage, the C═O is a carbonyl group of the phage, F¹ represents the labelling moiety, and N is a nitrogen of an amino group of the labelling moiety.

4. The LDB of claim 1, wherein the amide group or groups comprise 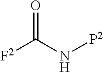
wherein P² represents the phage, N is a nitrogen of an amino group of the phage, F² represents the labelling moiety, and C═O is a carbonyl group of the labelling moiety.

5. The LDB of claim 1, wherein the amide group or groups comprise a linker of formula 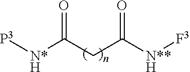
wherein P³ represents the phage, N* is a nitrogen of an amino group of the phage, F³ represents the labelling moiety, N** is a nitrogen of an amino group of the labelling moiety, and n is 1, 2, 3, 4, 5, 6, 7, 8, 9, or 10.

6. The LDB of claim 1, wherein the labelling moiety comprises one or more fluorescent moieties that are Atto Dye 590, Atto Dye 594, an amine-functionalized quantum dot, a carboxylate-functionalized quantum dot, fluorescein isothiocyanate (FITC), 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); VIC®, or a combination of any two or more thereof.

7. The LDB of claim 1, wherein the amide group or groups comprise a lysine residue of the phage.

8. A method for generating the LDB of claim 1, wherein the method comprises
contacting a phage comprising a carboxyl group disposed on the exterior surface of the capsid, a first activating agent, and a first carbodiimmide in the presence of a first solvent to provide a first activated species; and
subsequently contacting a first labelling moiety comprising an amine group with the first activated species to provide a first LDB;
wherein the first solvent comprises water.

9. A method for identifying at least one bacterial strain or species in a biological sample obtained from a subject comprising
(a) contacting the biological sample with an effective amount of the LDB of claim 1; and
(b) detecting the presence of LDB-infected bacterial cells, thereby leading to the identification of at least one bacterial strain or species in the biological sample.

10. A method for determining the antibiotic susceptibility of a bacterial strain or species in a biological sample obtained from a subject comprising
(a) contacting a plurality of test samples comprising bacterial cells with an effective amount of the LDB of claim 1 and at least one antibiotic, wherein the plurality of test samples is derived from the subject;
(b) detecting the presence of LDB-infected bacterial cells in the plurality of test samples; and
(c) determining that an antibiotic is effective in inhibiting the bacterial strain or species when the number of LDB-infected bacterial cells in the test sample is reduced relative to that observed in an untreated control sample comprising bacterial cells, wherein the untreated control sample is derived from the subject.

11. The method of claim 9, wherein the LDB further comprises at least one reporter molecule that is intercalated within the nucleic acids of the LDB, wherein the at least one reporter molecule intercalated within the nucleic acids of the LDB is selected from the group consisting of SYBR Gold, SYBR Green I, SYBR Safe, Quant-iT PicoGreen, Blue-Fluorescent SYTO dyes (e.g., SYTO 40, SYTO 41, SYTO 42, SYTO 45), Green-Fluorescent SYTO dyes (e.g., SYTO 9, SYTO 10, SYTO BC, SYTO 13, SYTO 16, SYTO 24, SYTO 21, SYTO 12, SYTO 11, SYTO 14, SYTO 25), Orange-Fluorescent SYTO dyes (e.g., SYTO 81, SYTO 80, SYTO 82, SYTO 83, SYTO 84, SYTO 85), Red-Fluorescent SYTO dyes (e.g., SYTO 64, SYTO 61, SYTO 17, SYTO 59, SYTO 62, SYTO 60, SYTO 63), cyanine dimer dyes (e.g., TOTO-1, YOYO-1, POPO-1, LOLO-1, BOBO-1, JOJO-1, POPO-3, BOBO-3, YOYO-3, TOTO-3), and other cyanine dyes, Acridine homodimer, Acridine orange, 7-AAD, ACMA, DAPI, Dihydroethidium, ethidium bromide, EthD-1, EthD-2, Hoechst 33258, Hoechst 33342, Hoechst 34580, hydroxystilbamidine, and LDS 751.

12. The method of claim 9, wherein the LDB further comprises at least one reporter molecule that is encoded by a heterologous nucleic acid located within the genome of the LDB, wherein the at least one reporter molecule encoded by the heterologous nucleic acid is a fluorescent label, a luminescent label, a colorimetric label, an electrochemical label, or a mechanical label.

13. The method of claim 12, wherein the at least one reporter molecule encoded by the heterologous nucleic acid is captured on a microbead or a solid support microstructure.

14. The method of claim 13, wherein the at least one reporter molecule encoded by the heterologous nucleic acid comprises an affinity domain that specifically binds to the microbead or the solid support microstructure.

15. The method of claim 13, wherein the microbead or solid support microstructure is coded to facilitate the identification of a specific bacteria strain or species in the biological sample.

16. The method of claim 13, wherein the microbead or the solid support microstructure is coated with a reagent that allows capture of the at least one reporter molecule produced by LDB-infected bacterial cells.

17. The method of claim 9, furthering comprising contacting the LDB-infected bacterial cells with a microbead or a solid support microstructure that is optionally coated with a reagent that allows capture of the LDB-infected bacterial cells.

18. The method of claim 17, wherein the microbead or the solid support microstructure is captured in a microwell array that is optionally sealed by mechanical sealing or oil sealing, wherein the microwell array contains 1 bead per well or more than 1 bead/well.

19. The method of claim 17, wherein the microbead or the solid support microstructure is spread on a surface that does not contain microwells.

20. The method of claim 9, wherein the biological sample comprises no more than 10 bacterial cells/ml.
